(19) United States
(12) Patent Application Publication
(10) Pub. No.: US 2011/0129849 A1
(43) Pub. Date: Jun. 2, 2011

(54) DETECTION OF PROSTATE CANCER USING PSA GLYCOSYLATION PATTERNS

(75) Inventors: Hui Zhang, Elicott City, MD (US); Danni Li Meany, Baltimore, MD (US); Daniel W. Chan, Clarksville, MD (US); Zhen Zhang, Dayton, MD (US); Yan Li, Middle River, MD (US); Lori J. Sokoll, Owings Mills, MD (US)

(73) Assignee: THE JOHNS HOPKINS UNIVERSITY, Baltimore, MD (US)

(21) Appl. No.: 13/055,868

(22) PCT Filed: Jul. 27, 2009
(86) PCT No.: PCT/US09/04365

§ 371 Date: Jan. 25, 2011

Related U.S. Application Data
(60) Provisional application No. 61/083,642, filed on Jul. 25, 2008.

(51) Int. Cl. G01N 33/574

(52) U.S. Cl. 435/7.1

(57) ABSTRACT

The present invention features novel methods for determining if a subject has prostate cancer. The present invention is based on the development of lectin immunosorbant assays which analyze α2,6-linked sialylation of total serum PSA by sambucus nigra lectin (SNA) and α2,3-linked sialylation of total and free serum PSA. These novel assays were used then to conduct a clinical investigation of the potential role of glycoprotein analysis in improving PSA's cancer specificity. The present invention also features kits for determining if a subject has prostate cancer comprising one or more lectins and a PSA specific antibody and instructions for use.

### CROSS-REFERENCE TO RELATED APPLICATION

This application claims the benefit of U.S. Provisional Application No. 61/083,642, which was filed Jul. 25, 2008, the entire contents of which are incorporated herein by reference.

### BACKGROUND OF THE INVENTION

In the United States, prostate cancer is the most common malignancy in men and the second leading cause of death from cancer. Each year over 300,000 men are diagnosed with prostate cancer in the U.S. alone. Both the incidence of prostate cancer and its associated mortality have been increasing over the past ten years. Recently, it has been shown that women with breast cancer also exhibit PSA. PSA production in breast tumors is associated with estrogen and/or progesterone receptor presence. Typically, PSA levels in female serum are undetectable.

Currently, prostate-specific antigen (PSA) is the best tumor marker available for the early detection of prostate cancer. However, PSA lacks specificity as it can be elevated in men with cancer as well is in men with benign prostate conditions. The typically used assay cutoff for PSA is 4.0 ng/mL, although lower cutoffs of 2.0 ng/mL, 2.5 ng/mL and 2.8 ng/mL have been suggested as it is recognized that there is risk for prostate cancer over all ranges of PSA. Men with total PSA between 4 and 10 ng/mL are in a diagnostic gray zone of total PSA, in which a biopsy would reveal no evidence of cancer in three out of four men, which results in a number of unnecessary biopsies.

Glycosylation is one of the most universal post-translational modifications of proteins, and it is involved in protein interactions, cell-cell recognition, adhesion, and motility. Recently, increasing evidence suggests that cell surface glycosylation is altered in disease states such as cancer, which indicates that glycosylation is associated with disease development. Accordingly, glycosylation patterns of the glycoproteins may be expected to improve the specificity of disease diagnosis. For example, PSA is a serum marker which has been approved by the Food and Drug Administration (FDA) for prostate cancer screening and monitoring. However, PSA alone is not specific enough to distinguish the early stage cancer for all cases, especially in the “diagnostic grey zone” of the PSA concentration from 4 to 10 ng/mL in serum. PSA has been reported as a glycoprotein which has an N-oligosaccharide chain attached to Asn-45. In addition to PSA protein level, the change of PSA carbohydrate structure could be used to distinguish the PSA from normal and cancer origins. Consequentially, the glycosylation patterns of PSA have the potential to be used as the new biomolecular markers for cancer detection when the PSA protein level cannot distinguish normal and cancer groups.

In serum, the majority of total PSA is complexed with antiproteases, whereas 5 to 45% is in a free, uncomplexed form. In an attempt to improve the cancer specificity of PSA in its diagnostic gray zone, it was discovered that men with prostate cancer have a lower ratio of free to total PSA compared to men without prostate cancer. Consequently, percent free PSA (% free PSA) is recommended for risk assessment for prostate cancer when total PSA concentrations are between 4-10 ng/mL. A percent (%) free PSA of >25% indicates a lower risk of cancer (e.g. probability=8%) whereas a % free PSA of <10% suggests a higher risk (e.g. probability=56%). However, the majority of patients tested for % free PSA fall into the midrange (e.g. 10-20%) for whom the risk of cancer is about 25%, hence, another diagnostic gray zone. The knowledge that free PSA is composed of both cancer-specific (e.g., [−2]proPSA) and benign-specific (e.g., BPSA) forms explains the limitation of % free PSA.

Accordingly, there is a need in the art for improved methods for prostate cancer detection.

### SUMMARY OF THE INVENTION

As described below, the present invention features novel methods for determining if a subject has prostate cancer. The present invention is based on the development of lectin immunosorbant assays (total SNA, total MAL I, free MAL I, total MAL II, and free MAL II), which analyze α-2,6-linked sialylation of total serum PSA by sambucus nigra lectin (SNA) and α2,3-linked sialylation of total and free serum PSA. These novel assays were used then to conduct a clinical investigation of the potential role of glycoprotein analysis in improving PSA's cancer specificity.

Accordingly, in a first aspect, the invention features a method of determining if a subject has prostate cancer comprising determining if the subject has an altered prostate specific antigen (PSA) glycosylation pattern as compared to the glycosylation pattern of PSA from a healthy subject wherein an altered glycosylation pattern is indicative that the subject has prostate cancer.

In one embodiment, the glycosylation pattern is α2,3-linked sialylation or α2,6-linked sialylation of PSA.

In another embodiment of any one of the above aspects, the PSA glycosylation pattern is determined by one or more lectin immunosorbant assays. In a further embodiment, the one or more lectin immunosorbant assays sandwich serum PSA between a PSA antibody and one or more lectins.

In another further embodiment, the one or more assays are selected from the group consisting of total PSA with SNA, total PSA with MAL I, total PSA with MAL II, free PSA with MAL I and free PSA with MAL II.

In a further embodiment, the method comprises at least 2 lectin immunosorbant assays. In another further embodiment, the method comprises at least 3 lectin immunosorbant assays. In still another further embodiment, the method comprises at least 4 lectin immunosorbant assays. In another related embodiment, the method comprises 5 lectin immunosorbant assays.

In one embodiment, the method of any one of the above aspects further comprises isolating PSA from a biological sample using a PSA specific antibody.

In another embodiment, the PSA specific antibody is specific for free PSA.

In another embodiment, the PSA specific antibody is specific for total PSA.

In certain embodiments, the antibody is treated to remove the binding of one or more glycans from the antibody to lectin prior to use. In further related embodiments, the treatment is oxidation. In a further embodiment of any one of the above aspects, the antibody is oxidized prior to use.

In another embodiment, the antibody is preferably oxidized with sodium periodate.

In another embodiment of any one of the above aspects, the subject is preselected based on the levels of free PSA. In another related embodiment of any one of the above aspects, the subject is preselected based on the levels of total PSA.

In a further embodiment, the level of free PSA is between about 10% and about 25%.

In another further embodiment, the level of total PSA is between about 2-10 ng/ml.

In another embodiment of any one of the above aspects, the altered PSA glycosylation pattern is a more heterogeneous pattern in subjects having cancer.

In another aspect, the invention features a method of determining if a subject has prostate cancer, comprising determining if a subject has an altered PSA α2,6-sialylation pattern as compared to the α2,6-sialylation pattern of PSA from a healthy subject, wherein an altered PSA α2,6-sialylation pattern is indicative of prostate cancer.

In one embodiment, the PSA α2,6-sialylation pattern is determined by lectin immunosorbant assay.

In another embodiment, the lectin immunosorbant assay is an assay of total PSA with SNA.

In still another further embodiment, the method further comprises isolating total PSA from a biological sample using a total PSA specific antibody.

In another embodiment, the subject is preselected based on the levels of free PSA.

In a further embodiment, the level of free PSA is between about 10% and about 25%.

In another embodiment, the subject is preselected based on the levels of total PSA.

In a further embodiment, the level of total PSA is between about 2-10 ng/ml.

In another particular embodiment, the antibody is treated to remove the binding of one or more glycans from the antibody to lectin prior to use. In a related embodiment, the treatment is oxidation. In another embodiment, the antibody is oxidized prior to use. In a related embodiment, the antibody is oxidized with sodium periodate.

In another aspect, the invention features a method of determining if a subject has prostate cancer, comprising determining if a subject has an altered PSA α2,3-sialylation pattern as compared to the α2,3-sialylation pattern of PSA from a healthy subject, wherein an altered PSA α2,3-sialylation pattern is indicative of prostate cancer.

In one embodiment, the PSA α2,3-sialylation pattern is determined by lectin immunosorbant assay.

In another embodiment, the lectin immunosorbant assay is an assay of total PSA with SNA.

In another embodiment, the method further comprises isolating total PSA from a biological sample using a total PSA specific antibody.

In a further embodiment, the subject is preselected based on the levels of free PSA. In a related embodiment, the level of free PSA is between about 10% and about 25%.

In another embodiment, the subject is preselected based on the levels of total PSA. In a further embodiment, the level of total PSA is between about 2-10 ng/ml.

In another particular embodiment, the antibody is treated to remove the binding of one or more glycans from the antibody to lectin prior to use. In a related embodiment, the treatment is oxidation.

In another embodiment, the antibody is oxidized prior to use. In a related embodiment, the antibody is oxidized with sodium periodate.

In another aspect, the invention features a method for determining if a subject has cancer or benign prostate hyperplasia (BPH) comprising determining if a subject has an altered PSA α2,6-sialylation pattern as compared to the α2,6-sialylation pattern of PSA from a healthy subject, wherein an altered PSA α2,6-sialylation pattern is indicative of prostate cancer and a non-altered α2,6-sialylation pattern of PSA is indicative of benign prostate hyperplasia.

In one embodiment, the subject was previously determined to have either cancer of BPH.

In another embodiment, the PSA α2,6-sialylation pattern is determined by lectin immunosorbant assay.

In a further embodiment, the lectin immunosorbant assay is an assay of total PSA with SNA.

In a related embodiment, the method further comprises isolating total PSA from a biological sample using a total PSA specific antibody.

In another particular embodiment, the antibody is treated to remove the binding of one or more glycans from the antibody to lectin prior to use. In a related embodiment, the treatment is oxidation.

In another embodiment, the antibody is oxidized prior to use. In a related embodiment, the antibody is oxidized with sodium periodate.

In another aspect, the invention features a method of determining if a subject has prostate cancer comprising determining if a sample of PSA from a subject has increased levels of glycosylation with sialic acid, O-linked galactose or Man/GlcNAc with Fuca1-6 groups as compared to PSA from a healthy subject, wherein PSA with increased levels of glycosylation with sialic acid, O-linked galactose or Man/GlcNAc with Fuca1-6 groups as compared to PSA from a healthy subject is indicative of prostate cancer.

In one embodiment, the PSA glycosylation pattern is determined by one or more lectin immunosorbant assays.

In another embodiment, any one of the above methods further comprises isolating PSA from a biological sample using a PSA specific antibody.

In one embodiment, the PSA specific antibody is specific for free PSA.

In another embodiment, the PSA specific antibody is specific for total PSA.

In another particular embodiment, the antibody is treated to remove the binding of one or more glycans from the antibody to lectin prior to use. In a related embodiment, the treatment is oxidation.

In another embodiment of any one of the above methods, the antibody is oxidized prior to use. In a related embodiment, the antibody is oxidized with sodium periodate.

In still another embodiment of any one of the above methods, the subject is preselected based a family history of cancer.

In a related embodiment, glycosylation with sialic acid is determined using lectin SNA-1.

In another related embodiment, glycosylation with O-linked galactose is determined using lectin Jacalin. In a further embodiment, glycosylation with Man/GlcNAc with Fuca1-6 groups is determined using lectin LcH.

In another aspect, the invention features a kit for determining if a subject has prostate cancer comprising one or more lectins and a PSA specific antibody and instructions for use.

In one embodiment, the lectins are selected from the group consisting of SNA, MAL I, and MAL II. In a related embodiment, the lectins are further selected from Jacalin and LcH.

In a further embodiment, the PSA specific antibody is specific for free PSA. In another further embodiment, the PSA specific antibody is specific for total PSA. In a related embodiment, the antibody is oxidized. In another further embodiment, the antibody is oxidized with sodium periodate.

In another aspect, the invention features a kit for determining if a subject has prostate cancer comprising an antibody specific for total PSA and a lectin that is specific for α2,6-sialylation, and instructions for use.

In one embodiment, the antibody is oxidized. In a further related embodiment, the antibody is oxidized with sodium periodate.

In still another aspect, the invention features a kit for determining if a subject has prostate cancer comprising lectins SNA-1, Jacalin, and LcH, a PSA specific antibody and instructions for use.

In one embodiment, the antibody is oxidized. In a further related embodiment, the antibody is oxidized with sodium periodate.

Other features and advantages of the invention will be apparent from the detailed description, and from the claims.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 is a graph that shows binding curves of five lectin immunosorbant assays for total or free PSA.

FIG. 2 (A-E) is a panel of graphs that show comparison of the sialylation of total and free PSA between 3 prostate cancer serum pools and 3 non-cancer serum pools by total SNA (A), total MAL I (B), free MAL I (C), total MAL II (D), and free MAL II (E) assays. Pool 1 in the cancer and non-cancer groups were measured 21 times whereas pools 2 and 3 were measured 3 times. These six pools have matched total PSA and free PSA levels: total PSA concentrations in pool 1, 2, 3 of the cancer and non-cancer groups are 5.26, 5.04, 5.92, 5.20, 5.03, and 4.94 ng/mL, respectively; free PSA concentrations are 0.98, 0.84, 1.15, 1.13, 1.61, and 0.80 ng/mL, respectively.

FIG. 3 (A-C) is three graphs that show ROC analysis of the cancer and non-cancer groups in (A) all 52 subjects with free PSA in the 4.7-31.8% range, (B) in a subset of 21 subjects with free PSA in the 10-20% range, and (C) in a separate study of 16 subjects with free PSA of 10-20% range.

FIGS. 4 (A and B) shows the detection of glycosylation pattern of human seminal fluidic PSA using high-density lectin microarray.

FIG. 5 are two graphs that show the binding curves of two lectin candidates of the developed immunoassays.

FIG. 6 are two graphs that show validation of targeted glycan-lectin bindings using developed ECL-based immunoassays in prostate tissue samples.

### DETAILED DESCRIPTION OF THE INVENTION

### I. Definitions

Unless defined otherwise, all technical and scientific terms used herein have the meaning commonly understood by a person skilled in the art to which this invention belongs.

The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Unless otherwise specified, “a” or “an” means “one or more”.

Unless specifically stated or obvious from context, as used herein, the term “or” is understood to be inclusive.

The term “antibody” is meant to refer to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The term “glycosylation pattern” as used herein is meant to refer to the presentation of glycan structures (oligosaccharides) present in a pool of PSA. A glycoprofile can be presented, for example, as a plurality of peaks each corresponding to one or more glycan structures present in a pool of PSA.

The term “lectin immunosorbant assay” is meant to refer to an immunochemical test that involves a lectin and an antibody or antigen. In a preferred embodiment, the immunosorbant assays are meant to refer to an immunosorbant assay that to sandwiches serum PSA between a PSA antibody and one or more lectin. In particular preferred embodiments, lectins can either be used to detect the glycosylation changes of PSA captured by PSA Ab or can be used to bind PSA followed by detection by PSA Ab.

The term “prostate-specific antigen (PSA)” is meant to refer to a 33 kDa chymotrypsin like protein that is a member of the human kallikrein gene family. In preferred embodiments, PSA is a protein produced by cells of the prostate gland.

The term “sample” is meant to refer to any bodily fluid or tissue from a subject, including but not limited to urine, blood, serum, semen, saliva, feces, or tissue. A sample as used herein can be unconcentrated or can be concentrated using standard methods.

The term “sialylated” or “sialylation” refers to covalent modification by one or more sialylic acid moieties. In certain embodiments, sialylation is of PSA. In certain embodiments, sialylation can be a 2, 6 linked sialylation of PSA. In other embodiments, sialylation can be a 2, 3 linked sialylation of PSA.

The term “subject” is meant to refer to an animal, more preferably a mammal, and most preferably a human.

Each patent, patent application, or reference cited herein is hereby incorporated by reference as if each were incorporated by reference individually.

### Prostate Specific Antigen

Prostate-specific antigen (PSA), also known as also known as human kallikrein III (hk3), seminin, semenogelase, gamma-seminoprotein, and P-30, is a member of the human kallikrein gene family, 33 kDa chymotrypsin like protein that is synthesized exclusively by normal, hyperplastic, and malignant prostatic epithelia. PSA's tissue-specific relationship has made it an attractive biomarker for identifying benign prostatic hyperplasia (BPH) and prostatic carcinoma (CaP) or metastatic cancer. Normal serum levels of PSA and blood are typically below 5 ng/ml, with elevated levels indicative of BPH or CaP. For example, serum levels of 200 ng/ml have been measured in end-stage metastatic CaP. The typically used assay cutoff for PSA is 4.0 ng/mL, 1 although lower cutoffs of 2.0 ng/mL, 2.5 ng/mL and 2.8 ng/mL have been suggested as it is recognized that there is risk for prostate cancer over all ranges of PSA

Prostate specific antigen (PSA) is most commonly known as a protein produced by the epithelial cells of the prostate gland. PSA is present in small quantities in the serum of normal men, and is often elevated in the presence of prostate cancer or other prostate disorders. Currently, a blood test is used to measure PSA levels as a method of early detection of prostate cancer. Higher than normal levels of PSA are associated with both localized and metastatic prostate cancer.

In addition to seminal fluid, the presence of PSA has been demonstrated in salivary glands, pancreas, breast (healthy breast tissues and breast tumors, breast cystic disease), various breast secretions (nipple aspirate fluid, milk of lactating women), periurethral gland, endometrial tissue, amniotic fluid, bronchoalveolar washing, ascitic fluid, plueral effusions, and cerebrospinal fluid. Very low levels of PSA are detectable in female sera. PSA has also been detected in a variety of tumors including, ovarian tumors, thyroid neoplasm, bile duct neoplasm, lung neoplasm, bladder neoplasm, sweat gland neoplasm, paraurethral gland neoplasm, salivary gland neoplasm, pancreas neoplasm, kidney, colon and liver neoplasm.

PSA is normally present in the blood at very low levels; normal PSA levels are defined as between zero (0) to four (4) ng/ml. Increased levels of PSA may suggest the presence of prostate cancer in men or breast or other cancers in women. Most PSA in the blood is bound to serum protein. A small amount of PSA is not bound to serum protein. PSA in this form is called free PSA.

### Methods

The present invention provides diagnostic or prognostic tests. In preferred aspects, the present invention provides a method of determining if a subject has prostate cancer comprising determining if the subject has an altered PSA glycosylation pattern as compared to the glycosylation pattern of PSA from a healthy subject, wherein an altered glycosylation pattern is indicative that the subject has prostate cancer. In particular embodiments, the methods described herein are particularly useful for determining if subjects who fall in they diagnostic “gray zone” using current methodology have prostate cancer.

As used herein, the term “gray zone” means the particular test values wherein a clear diagnosis of cancer or cancer-free can be made.

Aberrant glycosylation has been reported in essentially all types of experimental and human cancer. Among others, changes in beta 1,6 GlcNAc branching structure and in the order of N-linked glycans, changes in sialylation of O-linked TN-antigen and changes in expression levels of sialylated and unsialylated Lewis factors have all been correlated to tumor progression.

In general, the carbohydrate moiety of any N-linked glycoprotein can be placed in one of three major categories on the basis of the structure and location of the monosaccharide added to this trimannosyl core: high mannose, hybrid or complex. For all of these structures, the link to the protein is through the amino acid asparagine (N-linked). In N-linked sugars the reducing terminal core is strictly conserved (Man3GlcNAc2) and the glycosylamine linkage is always via a GlcNAc residue. The large diversity of N-linked oligosaccharides arises from variations in the oligosaccharide chain beyond the core motif. First, there can be differential extension of the biantennary arms of the core. Second, variation can arise from increased branching resulting in tri- and tetrantennary structures. In this case, several N-acetylglucosaminyl transferases can act on the biantennary structure to form more highly branched oligosaccharides.

O-linked glycans attach to proteins by an O-glycosidic bond to serine or threonine on the peptide chain. Unlike N-linked sugars, O-linked sugars are based on a number of different cores, giving rise to great structural diversity. O-linked glycans are generally smaller than N-linked, and there is no consensus motif for locating O-linked glycosylation on the protein.

Changes in glycosylation patterns are known to alter the specificity and/or structure of proteins and as a consequence their function, and changes in glycosylation have been long thought to be markers of tumor progression.

Glycosylation pattern is meant to refer to the presentation of glycan structures (oligosaccharides) present in a pool of PSA. A glycoprofile can be presented, for example, as a plurality of peaks each corresponding to one or more glycan structures present in a pool of PSA.

In preferred embodiments of the present method, the glycosylation pattern of PSA from a subject is compared to the glycosylation pattern of PSA from a healthy subject. For example, the subject can be a subject that has a disease that is compared to a control subject that does not have the disease. Patterns of PSA glycosylation that are different between the two samples can be used as biomarkers of disease, e.g., for diagnostic purposes, and are candidates for drug targets. Such biomarkers can also be used to monitor the response of a subject to a therapy, e.g., drug therapy.

The methods of the present invention have a number of applications, for example, detecting changes in patterns of PSA glycosylation over time; detecting interindividual patterns of PSA glycosylation activation or inactivation; development of diagnostics; identification of biomarkers for drug discovery and development; therapeutic glycoprotein development (e.g., to monitor process changes, process qualification/validation, trials); or in the purification of a glycoprotein therapeutic. A biomarker can be a single marker or a glycoprotein profile or glycoprotein pattern change.

PSA glycosylation pattern can be determined through immunosorbant assays. Preferably, the PSA glycosylation pattern is determined by one or more lectin immunosorbant assays. At least 160 lectins are known in the art. Examples include, but are not limited to, Maackia amurensis lectin I (MAL I) Maackia amurensis lectin II (MAL II), Sambucus nigra lectin (SNA, EBL) Concanavalin A (Con A), wheat germ agglutinin (WGA), Jacalin lectin (Jacalin), Aleuria aurantia lectin (AAL), Hippeastrum hybrid lectin (HHL, AL), Ulex europaeus Agglutinin I (UEA I), Lotus tetragonolobus lectin (LTL), and Galanthus nivalis lectin (GNL). Commercial sources of lectins include Vector Laboratories, Inc. (Burlingame, Calif.), GALAB Technologies (Geesthacht, Germany), and Sigma (St. Louis, Mo.). Alternatively, lectins can be isolated from natural sources or synthesized.

In particular preferred embodiments, the one or more lectin immunosorbant assays are selected from total PSA with SNA, total PSA with MAL I, total PSA with MAL II, free PSA with MAL I, free PSA with MAL II, PSA with LcH, PSA with SNA-1, and PSA with Jacalin.

In particular embodiments, for example, antibodies, such as a PSA antibody, preferably a PSA monoclonal antibody, may be immobilized onto a selected surface, preferably a surface exhibiting a protein affinity such as the wells of a polystyrene microtiter plate and incubated overnight. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation. In certain exemplary embodiments, in order to prevent binding of lectins to the carbohydrate determinants on the PSA antibody, antibody coated on the plates is preferably treated with sodium periodate buffer.

Following formation of specific immunocomplexes between the test sample and the bound antibody, and subsequent washing, the occurrence and even amount of immunocomplex formation may be determined by subjecting same to a lectin having specificity for the target.

To provide a detecting means, the lectin will preferably have an associated enzyme that will generate a color development upon incubating with an appropriate chromogenic substrate, or for example will have a biotin label that is detectable with a streptavidin substrate. Thus, for example, one will desire to contact and incubate the biotin conjugated lectin with streptavidin for a period of time and under conditions which favor the development of complex formation (e.g., 1 hr at room temperature).

Electrochemiluminescence can be Used to Detect the Amount of Labeled Biotin Labeled PSA.

Determining altered PSA glycosylation, e.g. α2,6-sialylation or α2,3-sialylation may also be carried out by immunoblot or Western blot analysis. For example, PSA antibodies may be used as high-affinity primary reagents for the identification of proteins immobilized onto a solid support matrix, such as nitrocellulose, nylon or combinations thereof, and in conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel- or fluorescently-tagged secondary antibodies against particular lectins as described herein.

In certain preferred embodiments, the method comprises at least 2 lectin immunosorbant assays. In other preferred embodiments, the method comprises at least 3 lectin immunosorbant assays. In further preferred embodiments, the method comprises at least 4 lectin immunosorbant assays. In further preferred embodiments, the method comprises at least 5 lectin immunosorbant assays. Preferably, the method comprises as many lectin immunosorbant assays necessary to determine if a subject has prostate cancer.

Sialyl acids are nine-carbon carboxylated sugars which exist in three primary forms. “Sialylated” refers to covalent modification by one or more sialic acid moieties. The most common is N-acetyl-neuraminic acid (2-keto-5-acetamido-3,5-dideoxy-D-glyc-ero-D-galactononulopyranos-1-onic acid (often abbreviated as NeuSAc, NeuAc, or NANA). A second common form is N-glycolyl-neuraminic acid (Neu5Gc or NeuGc), in which the N-acetyl group of NeuAc is hydroxylated. A third primary sialic acid is 2-keto-3-deoxy-nonulosonic acid (KDN). Typically found at the reducing end of glycans attached to cell surfaces or plasma proteins, sialic acids are typically over expressed in tumor cells, relative to normal tissues. These terminal sialic acids are involved in cellular adhesion and are components of cell surface receptors. Excess sialylation may mask specific cellular recognition sites, which is an important component of physiological responses to cancer cells. Lewis X and Lewis A blood group antigens, which are sialic acid containing proteins, are also typically overexpressed in carcinomas. Additional qualitative and quantitative changes in tumor cell surface sialic acids are associated with progression to malignancy. Tumor cells can change the sialo-glyco-conjugates expressed on their plasma membranes, which affects their ability to invade. Quantitative and qualitative assessment of protein sialylation in biological samples is increasingly recognized as a valuable contribution to diagnosis, prognosis and monitoring of conditions associated with over-sialylation of proteins. Such conditions include diabetes and myeloma, epithelial, breast, ovarian, oral, gastrointestinal, prostate, endometrial, lung, colon, pancreatic, and thyroid cancers.

In preferred embodiments of the present invention, the glycosylation pattern is α2,3-linked sialylation or α2,6-linked sialylation of PSA.

Accordingly, the invention also features methods of determining if a subject has prostate cancer, comprising determining if a subject has an altered PSA α2,3-sialylation pattern as compared to the α2,3-sialylation pattern of PSA from a healthy subject wherein an altered PSA α2,3-sialylation pattern is indicative of prostate cancer.

The invention also features methods of determining if a subject has prostate cancer, comprising determining if a subject has an altered PSA α2,6-sialylation pattern as compared to the α2,6-sialylation pattern of PSA from a healthy subject wherein an altered PSA α2,6-sialylation pattern is indicative of prostate cancer.

The methods can be carried out, for example, using the immunosorbant assays described herein.

In preferred embodiments, the sialylation pattern, in particular the pattern of PSA α2,3-sialylation or PSA α2,6-sialylation, is determined by lectin immunosorbant assay. Preferably, the lectin immunosorbant assay is and assay of total PSA with SNA. Total PSA can be isolated from a biological sample using a total PSA specific antibody.

In some embodiments, PSA, alone or in combination with other markers or clinical signs, measured as described herein, is used to determine whether the tumor is no longer in remission. In some embodiments PSA, alone or in combination with other markers or clinical signs, measured as described herein, is used to determine the extent of the tumor. In the latter case, percent of free PSA may be compared to total PSA; the smaller the percentage of free PSA, the more likely the presence of prostate cancer.

The methods of the invention may also be used to determine benign from cancerous tissue.

For example, in other aspects of the invention, methods include determining if a subject has cancer or benign prostate hyperplasia (BPH) comprising determining if a subject has an altered PSA α2,6-sialylation pattern as compared to the α2,6-sialylation pattern of PSA from a healthy subject, wherein an altered PSA α2,6-sialylation pattern is indicative of prostate cancer and a non-altered α2,6-sialylation pattern of PSA is indicative of benign prostate hyperplasia.

In certain cases, the subject may have previously been determined to have either cancer or BPH.

The PSA α2,6-sialylation pattern can be determined using the antibodies and methods as described herein, for example by one or more lectin immunosorbant assays. In certain embodiments, the lectin immunosorbant assay is an assay of total PSA with SNA. In exemplary embodiments, total PSA is isolated from a biological sample using a total PSA specific antibody.

In other aspects, the invention features methods of determining if a subject has prostate cancer comprising determining if a sample of PSA from a subject has increased levels of glycosylation with sialic acid, O-linked galactose or Man/GlcNAc with Fuca1-6 groups as compared to PSA from a healthy subject, wherein PSA with increased levels of glycosylation with sialic acid, O-linked galactose or Man/GlcNAc with Fuca1-6 groups as compared to PSA from a healthy subject is indicative of prostate cancer.

Preferably, using these methods the PSA glycosylation pattern is determined by one or more lectin immunosorbant assays.

In further exemplary embodiments, the method further comprises isolating PSA from a biological sample using a PSA specific antibody. The PSA specific antibody may be specific for free PSA, or the PSA specific antibody is specific for total PSA.

Antibodies useful in the methods of the invention are described herein.

In certain embodiments, the subject is preselected based a family history of cancer.

In particular embodiments, glycosylation with sialic acid is determined using lectin SNA-1. In other particular embodiments, glycosylation with O-linked galactose is determined using lectin Jacalin. In other particular embodiments, glycosylation with Man/GlcNAc with Fuca1-6 groups is determined using lectin LcH.

Changes in glycosylation patterns of glycoproteins may be assayed in a subject with a disease compared to a healthy subject, to monitor the presence or progress of the disease; at different times in a healthy subject to monitor the possible appearance of a disease, for example prostate cancer; in a subject with a disease undergoing treatment, to assess the influence of the treatment on the disease; to assess the influence of treatment on the subject; and post-treatment, to monitor for any possible relapse of the disease. For example, the subject may be a subject with prostate cancer.

Changes in glycosylation pattern may be an indication that the patient has prostate cancer, or that a patient is no longer in remission.

In certain embodiments, the altered PSA glycosylation pattern is a more heterogeneous pattern in subjects having cancer.

### Subjects and Samples

The samples that are used in the methods as described herein may be any suitable sample. Preferably, the sample is a biological sample. For example, in some embodiments, the sample(s) will be blood, serum, or plasma. In some embodiments, the sample or series of samples are serum samples. The individual may be an animal, e.g., mammal, e.g., human.

The sample may be a single sample, or the sample may be a series of a series of samples. If a series of samples is taken, they may be taken at any suitable interval, e.g., intervals of minutes, hours, days, weeks, months, or years. When an individual is followed for longer periods, sample intervals may be months or years. Diagnosis, prognosis, or method of treatment may be determined from a single sample, or from one or more of a series of samples, or from changes in the series of samples, e.g., an increase in concentration at a certain rate may indicate a severe condition whereas increase at a slower rate or no increase may indicate a relatively benign or less serious condition. The rate of change may be measured over the course of hours, days, weeks, months, or years. Rate of change in a given individual may, in some cases, be more relevant than an absolute value. In other settings, a rise in values over a period of days, weeks, months or years in an individual can indicate ongoing and worsening condition or recurrence of cancer.

In some embodiments, at least one sample is taken at or near the time the individual presents to a health professional with one or more symptoms indicative of a condition that in which PSA levels are elevated, for example cancer. In addition prostate cancer and breast cancer have other molecular markers that are detectable in the blood. The detection of these markers, in addition to PSA, may give a more definitive diagnosis of a cancerous condition. Other molecular markers for prostate cancer are known in the art, and may include, but are not limited to prostate specific membrane antigen (PSMA), KIAA 18, KIAA 96, prostate carcinoma tumor antigen-1 (PCTA-1), prostate secretory protein (PSP), prostate acid phosphatase (PAP), human glandular kallekrein 2 (HK-2), prostate stem cell antigen (PSCA), PTI-1, CLAR1 (U.S. Pat. No. 6,361,948), PG1, BPC-1, prostate-specific transglutaminase, cytokeratin 15, semenogelin II, NAALADase, PD-41, p53, TCSF (U.S. Pat. No. 5,856,112), p300, actin, EGFR, and HER-2/neuprotein, as well as other markers that will be apparent to those of skill in the art.

In one embodiment, the methods described herein are preformed after one of a test for one of the above identified markers does not allow for a conclusive diagnosis.

In preferred embodiments of the invention, the subject is preselected based on the levels of free PSA. In certain preferred embodiments, the level of free PSA is between about 10% and about 25%.

In other examples, the subject is preselected based a family history of cancer.

### Antibodies

In certain embodiments of the invention, PSA is isolated from a biological sample using a PSA specific antibody. An antibody can be a naturally occurring antibody as well as a non-naturally occurring antibodies, including, for example, single chain antibodies, chimeric, bifunctional and humanized antibodies, as well as antigen-binding fragments thereof. In some embodiments, the antibody is specific for free PSA. In some embodiments, the antibody is specific for total PSA. In some embodiments, the antibody is specific for PSA complexes. In some embodiments, an antibody specific to one or more particular forms of PSA may be used, e.g., a binding partner to complexed PSA, free PSA, total PSA, etc. Mixtures of antibodies are also encompassed by the invention, e.g., mixtures of antibodies to the various forms of the PSA (free, complexed, etc.), or mixtures of mixtures. In certain embodiments, the antibody is oxidized prior to use. In particular, the antibody may preferably be oxidized with sodium periodate.

It will be appreciated that the choice epitope or region of PSA to which the antibody is raised will determine its specificity, e.g., for free PSA, for complexed PSA, and the like. In some embodiments, the antibody is specific to a specific amino acid region of PSA.

In some embodiments the antibody is a polyclonal antibody. Polyclonal antibodies are useful as binding partners.

Methods for producing antibodies are well established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). The antibodies used in the present methods may be obtained in accordance with known techniques, and may be monoclonal or polyclonal, and may be of any species of origin, including (for example) mouse, rat, rabbit, horse, or human, or may be chimeric antibodies. See, e.g., M. Walker et al., Molec. Immunol. 26:403 (1989). The antibodies may be recombinant monoclonal antibodies produced according to the methods disclosed in U.S. Pat. No. 4,474,893, or 4,816,567, and WO/1998/022509, which are herein incorporated by reference in their entirety. The antibodies may also be chemically constructed by specific antibodies made according to the method disclosed in U.S. Pat. Nos. 4,676,980 and 5,501,983, which is herein incorporated by reference in their entirety. Monoclonal and polyclonal antibodies to free and complexed PSA are also commercially available (Dako, Carpenteria, Calif., Scantibodies, Inc, Santee, Calif., BiosPacific, Emeryville, Calif.).

In some embodiments, the antibody is a mammalian, e.g., goat polyclonal anti-PSA, antibody. The antibody may be specific to specific regions of PSA. Capture binding partners and detection binding partner pairs, e.g., capture and detection antibody pairs, may be used in embodiments of the invention. Thus, in some embodiments, a heterogeneous assay protocol is used in which, typically, two binding partners, e.g., two antibodies, are used. One binding partner is a capture partner, usually immobilized on a solid support, and the other binding partner is a detection binding partner, typically with a detectable label attached. In some embodiments, the capture binding partner member of a pair is an antibody that is specific to all or substantially all forms of PSA. An example is an antibody, e.g., a monoclonal antibody, specific to free PSA, and PSA complexes. Thus, it is thought that the antibody binds to total PSA.

In some embodiments it is useful to use an antibody that cross-reacts with a variety of species. Such embodiments include the measurement of drug toxicity by determining, e.g., the release of PSA into the blood as a marker of cancer. A cross-reacting antibody allows studies of toxicity to be done in one species, e.g. a non-human species, and direct transfer of the results to studies or clinical observations of another species, e.g., humans, using the same antibody or antibody pair in the reagents of the assays, thus decreasing variability between assays.

### Kits

The invention further provides kits.

Certain preferred kits of the invention include kits for determining if a subject has prostate cancer comprising one or more lectins and a PSA specific antibody and instructions for use. Other preferred kits include kits for determining if a subject has prostate cancer comprising an antibody specific for total PSA and a lectin that is specific for α2,6-sialylation, and instructions for use. Other kits of the present invention include kits for determining if a subject has prostate cancer comprising lectins SNA-1, Jacalin, and Lai., a PSA specific antibody and instructions for use.

Binding partners, e.g., antibodies, solid supports, and fluorescent labels for components of the kits may be any suitable such components as described herein.

The kits may additionally include reagents useful in the methods of the invention, e.g., buffers and other reagents used in binding reactions, washes, buffers or other reagents for preconditioning the instrument on which assays will be run, and elution buffers or other reagents for running samples through the instrument.

Kits may include one or more standards, e.g., standards for use in the assays of the invention, such as standards of highly purified, PSA, or various fragments, complexes, and the like, thereof. Kits may further include instructions.

Preferably, the lectins are selected from the group consisting of SNA, MAL I, and MAL II. The PSA specific antibody can be specific for free PSA or can be specific for total PSA. In certain embodiments, the antibody is oxidized, for example with sodium periodate.

The following examples are offered by way of illustration and not by way of limiting the remaining disclosure.

### EXAMPLES

### Example 1

### Glycoproteomics for Prostate Cancer Detection: Changes in PSA Glycosylation Patterns

Currently, serum prostate-specific antigen (PSA) is used for the early detection of prostate cancer despite its low specificity in the range of 4 to 10 ng/mL. Because aberrant glycosylation is a fundamental characteristic of tumor genesis, one objective of the present work was to investigate whether changes in PSA glycosylation may be used to improve the cancer specificity of PSA.

The present studies describe the development of five lectin immunosorbant assays (total SNA, total MAL I, free MAL I, total MAL II, and free MAL II), which analyze α2,6-linked sialylation of total serum PSA by sambucus nigra lectin (SNA) and α2,3-linked sialylation of total and free serum PSA by both maackia amurensis lectin I and II (MAL I and II). These assays were then used to conduct a clinical investigation of the potential role of glycoprotein analysis in improving PSAs cancer specificity.

### Lectin Immunosorbant Assays

Table 1, shown below, summarizes the capture antibodies and the lectins used in the lectin immunosorbant assays as well as the carbohydrate moieties they recognize. Table 1 shows five lectin immunosorbant assays for direct analysis of PSA sialylation in serum.

TABLE 1

| | Capture | | | |
|---|---|---|---|---|
| Assay | Antibody | Lectin | Lectin Specificity | LOD (ng/mL) |
| Total SNA | Total PSA | SNA | 2,6 sialic acid | 1.35 |
| Total MAL I | Total PSA | MAL I | 2,3 sialic acid | 0.14 |
| Free MAL I | Free PSA | MAL I | 2,3 sialic acid | 0.32 |
| Total MAL II | Total PSA | MAL II | 2,3 sialic acid | 0.07 |
| Free MAL II | Free PSA | MAL II | 2,3 sialic acid | 0.04 |

SNA, isolated from Sambucus nigra bark, binds to the disaccharide structure of sialic acid in an α2,6-linkage to galactose (Knibbs et al. 1991). MAL I (also known as MAL, MAA or MAM) and MAL II (also known as MAH) are both isolated from Maackia amurensis seeds. MAL I binds to the trisaccharide structure of sialic acid in an α2,3-linkage to galactose which is then in a β1,4-linkage to N-acetylglucosamine, (Knibbs et al. 1991) whereas MAL II appears to bind only particular carbohydrate structures that contain α2,3-linked sialic acid, (Kawaguchi et al. 1974) although its specificity is not well defined.

### Analytical Performance

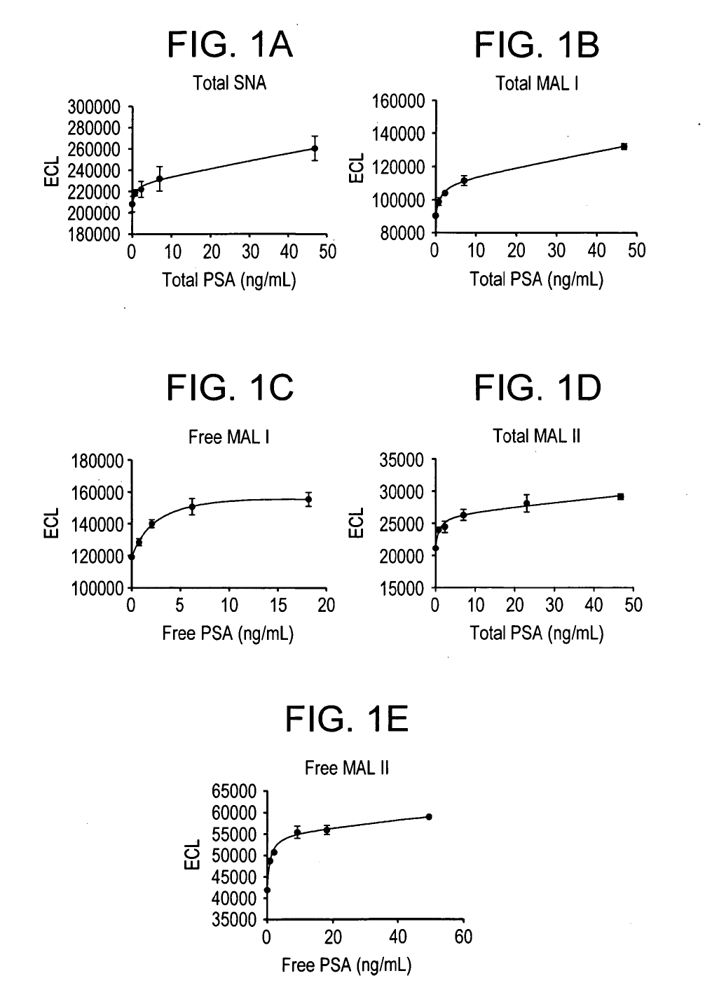

The binding curves of these five assays, established using pooled female sera spiked with human seminal fluid PSA, are shown in FIG. 1. It should be noted that the experiments can be carried out using pooled sera or in sera from individual serum samples (i.e. not pooled). Human seminal fluid PSA was used as the standard material because it harbors both α2,3-linked and α2,6-linked salic acid in its carbohydrate moiety. (Tabares et al., 2006; Peracula et al., 2003; Tajiri et al., 2008). In all these assays, the electrochemiluminescent signal increases with increasing concentrations of total PSA and free PSA as a result of the binding of lectins to carbohydrate on PSA molecules captured by the PSA antibody. The LOD of these five assays were calculated to be 1.35, 0.14, 0.32, 0.07, and 0.04 ng/mL of PSA, respectively (Table 1). They were well below the typically used assay cutoff for PSA (4.0 ng/mL) and therefore can be used in its diagnostic gray zone (4-10 ng/mL). In order to assess the within-run reproducibility of these assays, two male serum pools at two different endogenous total and free PSA concentrations were measured 27 times in a single run, as shown in Table 2, below. Table 2 shows within-run reproducibility (n=27) of five lectin immunosorbant assays determined using electrochemiluminescence intensity.

TABLE 2

| | Total SNA | | Total MAL I | | Free MAL I | | Total MAL II | | Free MAL II | |
|---|---|---|---|---|---|---|---|---|---|---|
| | Total PSA (ng/mL) | | Total PSA (ng/mL) | | Free PSA (ng/mL) | | Total PSA (ng/mL) | | Free PSA (ng/mL) | |
| | 4.12 | 11.22 | 4.12 | 11.22 | 0.91 | 0.99 | 4.12 | 11.22 | 0.91 | 0.99 |
| Mean | 331,804 | 349,333 | 60,872 | 81,598 | 81,509 | 100,350 | 19,613 | 24,328 | 26,096 | 29,267 |
| SD | 8636 | 7336 | 2,360 | 3,024 | 2,818 | 3,176 | 951 | 1,123 | 2,556 | 1,331 |
| % CV | 2.6 | 2.1 | 3.9 | 3.7 | 3.5 | 3.2 | 4.9 | 4.6 | 9.8 | 4.5 |

All five assays demonstrated excellent reproducibility, indicated by CVs less than 5% with the exception of a CV less than 10% for one of the free MAL II assays. The insignificant amount of non-PSA proteins present in the PSA standard (less than 2%) does not impact the assays or their capabilities to determine the carbohydrate moiety of PSA because i) a PSA antibody is used to capture PSA molecules from serum and ii) the binding curves were established using the total and free PSA concentrations measured by the Beckman ACCESS Hybritech PSA and Free PSA assays.

### Glycosylation Patterns of PSA Molecules In Sera

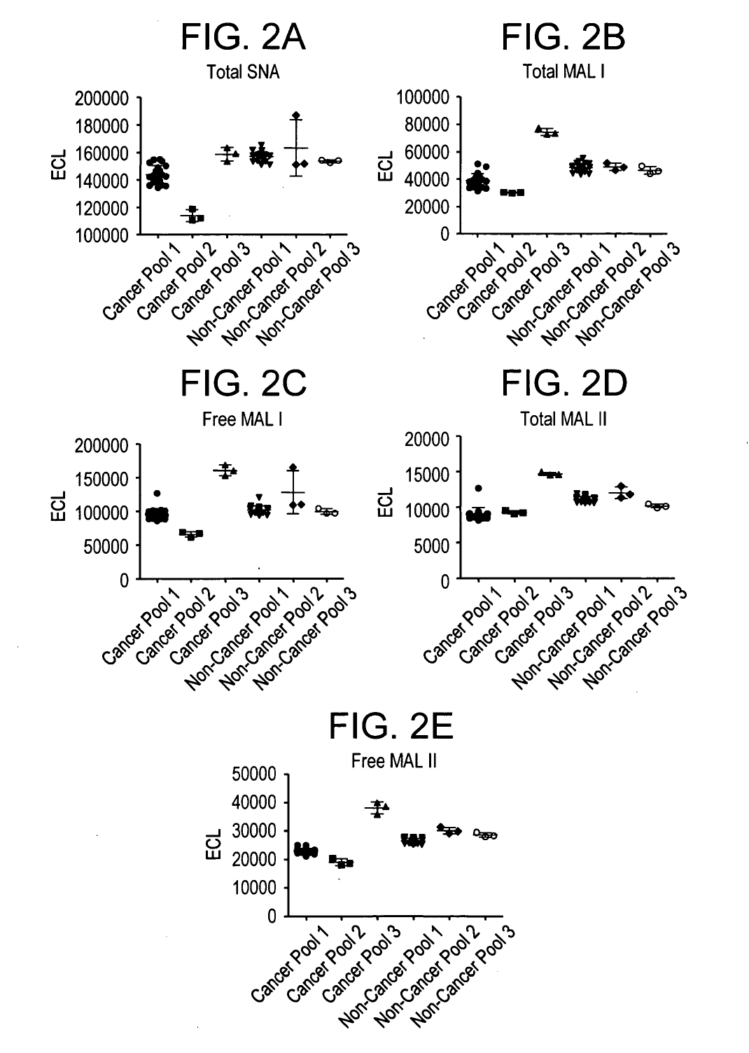

The sialylation patterns of free and total PSA molecules were compared in pooled sera between prostate cancer and non-cancer using the five lectin immunosorbant assays (FIG. 2). As noted above, it should be pointed out that the experiments can be carried out equally effectively using pooled sera or using serum samples from an individual (i.e. not pooled). Three pools of sera were prepared for each group to demonstrate their within-group and between-group similarities and differences. Given the limited number of samples that can be run on a 96-well plate, pool 1 in each group was measured 21 times whereas pools 2 and 3 were measured 3 times. A significant PSA sialylation pattern observed from this comparison was that prostate cancer sera showed relatively large within-group variation whereas non-cancer sera showed more consistent sialylation of PSA across the three pools, which may indicate a more heterogeneous sialylation pattern of PSA from cancer than non-cancer origins.

### Clinical Performance

Clinical performance of these assays was evaluated in 52 subjects with biopsy confirmed prostate cancer (n=26) or non-cancer (n=26). A comparison between the cancer and non-cancer groups for PSA concentrations, % free PSA, and the measured PSA glycosylation is shown in Table 3, shown below. Table 3 shows a comparison between the cancer (n=26) and non-cancer (n=26) groups for PSA concentrations, calculated % free PSA, and the measured PSA glycosylation.

TABLE 3

| | Prostate Cancer | | Non Cancer | | |
|---|---|---|---|---|---|
| | Mean ± SD | Median | Mean ± SD | Median | p Value |
| Total PSA (ng/mL) | 9.08 ± 5.16 | 8.42 | 7.65 ± 3.52 | 7.30 | 0.25 |
| Free PSA (ng/mL) | 0.89 ± 0.43 | 0.82 | 1.49 ± 0.86 | 1.54 | 0.0025 |
| % Free PSA | 10.97 ± 5.68 | 8.50 | 19.39 ± 6.85 | 20.17 | <0.001 |
| Total SNA^{a} | 178022 ± 46482 | 174335 | 186739 ± 38629 | 178223 | 0.47 |
| Total MAL I^{a} | 45011 ± 21952 | 44823 | 47605 ± 21737 | 43456 | 0.67 |
| Free MAL I^{a} | 49426 ± 23345 | 53635 | 53086 ± 23998 | 51038 | 0.58 |
| Total MAL II^{a} | 14382 ± 3369 | 14415 | 16000 ± 3896 | 15764 | 0.58 |
| Free MAL II^{a} | 16018 ± 3801 | 16111 | 17288 ± 4034 | 16023 | 0.11 |

Overall, Table 3 showed that the two study groups were not statistically different with respect to total PSA concentrations (p=0.25), but significantly different with respect to free PSA concentrations and % free PSA (p=0.0025 and p<0.001, respectively). Total SNA, total MAL I and MAL II were higher in the non-cancer group than in the cancer group, despite of the fact that total PSA concentrations in the cancer group were higher (cancer 9.08±5.16 ng/mL and non-cancer 7.65±3.52 ng/mL, mean±SD). This may suggest higher sialylation of total PSA in the non-cancer group than in the cancer group, although the differences were not statistically significant (p=0.47, 0.67, and 0.58, respectively).

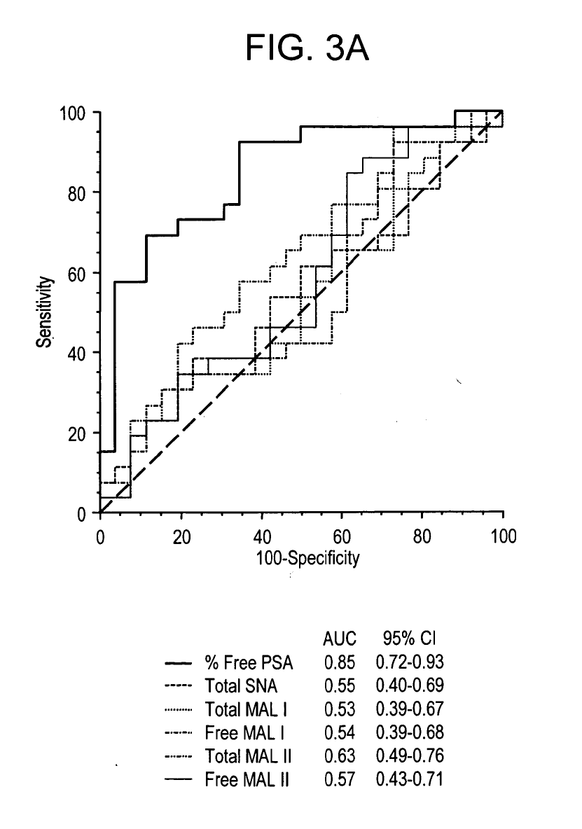

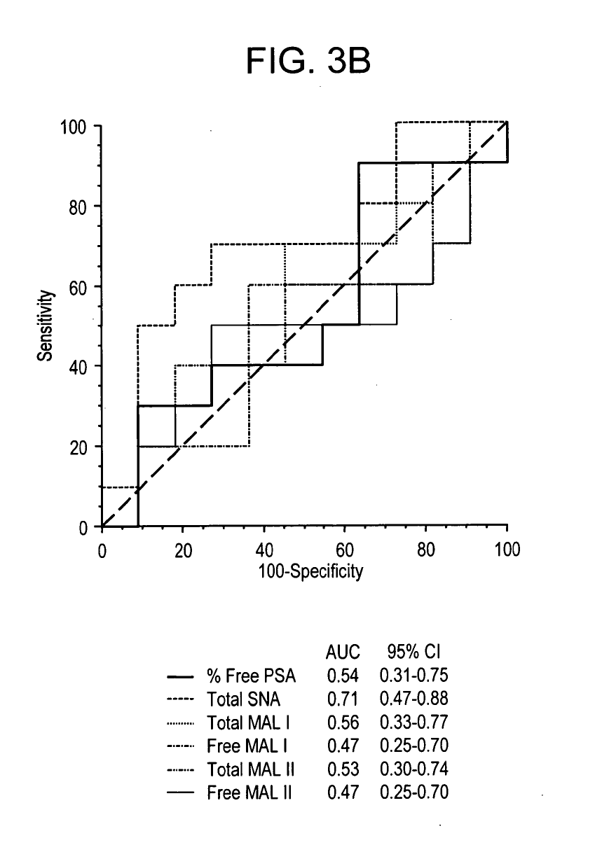

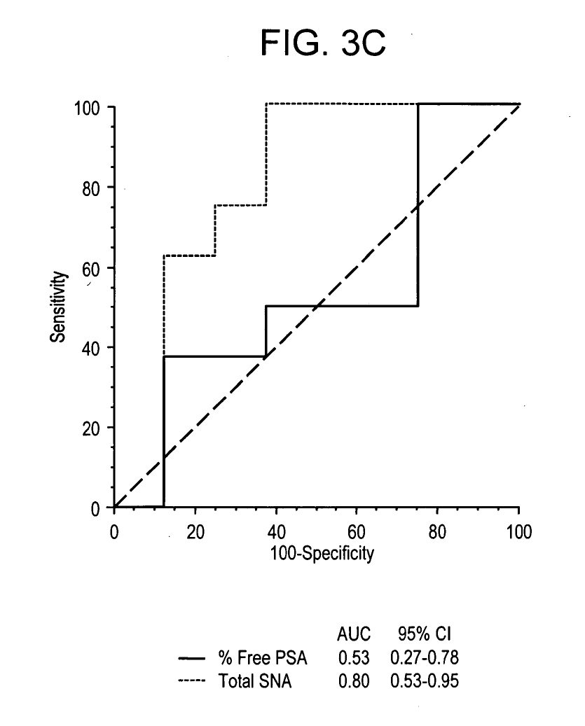

ROC analysis of the cancer and non-cancer groups in all 52 subjects (% free PSA in the 4.7-31.8% range) and in 21 subjects with % free PSA in the 10-20% range are shown in FIGS. 3A and 3B, respectively. % free PSA (AUC 0.85) was superior to all five assays (AUC 0.53-0.63) in all 52 subjects (p<0.05, FIG. 3A), However, in a subset of 21 subjects with % free PSA in the range of 10-20%, total SNA assay appeared to have a better clinical performance than % free PSA as shown by the AUCs (0.71 vs. 0.54, shown in FIG. 3B), although this difference was not statistically significant (p=0.27). In these 21 subjects, % free PSA was equivalent between the non-cancer (14.98±3.28%, mean±SD, n=11) and cancer (14.93±3.19%, n=10) groups, whereas the total SNA assay trended towards a higher average of 204713±40965 in the former than 170049±49060 in the latter (p=0.09). The other four lectin assays, however, did not show improvement over % free PSA in the 10-20% range (shown in FIG. 3B).

The improved performance trend of the total SNA assay over % free PSA in the 10-20% range was confirmed by applying the assay to a separate set of 16 subjects (8 prostate cancer and 8 non-cancer). Total PSA and % free PSA in the cancer (5.81±2.33 ng/mL and 14.53±3.20%) and non-cancer (4.98±1.47 ng/mL and 15.14±2.66%) groups were not statistically different (p=0.40 and 0.68, respectively). ROC analysis in these 16 subjects confirmed the improved performance trend of the total SNA assay compared to % free PSA (AUC 0.80 vs 0.53, FIG. 3C).

Although PSA is the best tumor marker available for prostate cancer, it is not perfect due to its lack of cancer specificity. % free PSA has improved PSA cancer specificity by the assessment of cancer risk from low to high using greater than 25% and less than 10% cutoffs, respectively. However, midrange % free PSA (10-20%) still presents a dilemma. (Sokoll et al. 2008). In fact, the majority of patients have a % free PSA in this midrange. Given that PSA is a 237-amino-acid single chain glycoprotein with 8.3% of its molecular weight carbohydrate, (Belanger et al. 1995) efforts for improvement have focused on searching for cancer-specific forms of PSA in both the amino-acid and carbohydrate portions. One example in the former is [−2]propSA, a truncated precursor form of PSA that has 2 additional amino acids in a pro-leader sequence. (Mikolajczyk et al. 2004). Recently an automated immunoassay for [−2]propSA has been developed and employed in a multi-center study, which showed that [−2]propSA was a better predictor of prostate cancer than % free PSA, particularly in the 2-10 ng/mL total PSA range. (Sokoll et al. 2008).

Although the search for glycosylated forms of PSA that may harbor cancer specificity began almost 20 years ago, (Barak et al. 1989; Chan et al. 1991) progress had been slow. Nevertheless, recent technological advances in glycan analysis renewed interest, particularly after recent publications illustrated different glycan structures of PSA from prostate cancer sera when compared to PSA from seminal fluid and non-cancer sera. (Tabares et al., 2006; Peracula et al., 2003; Tajiri et al., 2008). This suggested the development of clinically useful and direct assays to detect PSA glycosylation in serum may be promising.

The present invention describes the development of five lectin immunosorbant assays for direct analysis of PSA sialylation in serum. Lectin immunosorbant assays are similar to enzyme-linked immunosorbant assays (ELISA) except that lectins are used as probes for detecting glycan structures. (Lotan et al. 1979) Readily available in pure form, lectins have been extensively used as probes for glycan structures because 1) they have specificity towards mono- or oligosaccharides through complimentary sugar-binding sites and 2) they generally do not interact with protein backbones. However, lectin immunosorbant assays are only used in a small number of research laboratories for three reasons. First, antibodies used in these assays need to be deglycosylated, otherwise lectins would bind not only glycan on proteins captured by antibodies but also to glycans on antibodies, resulting in a high background (McCoy et al. 1983; Mehta et al. 2008; Gornik et al. 2007). Second, because binding affinities of lectins (ranging from 10⁶ to 5×10⁷M⁻¹) are 100- to 10,000-fold lower than those of antibodies (˜10⁸ to 10¹²M⁻¹) (Lotan et al., 1979; Davies et al. 1994) and analytes of interest usually have very low concentrations (˜ng/mL) in serum, the limit of detection of theses assays may be insufficient in ˜ng/mL ranges. Third, because lectins only have specificity to glycan but not proteins, they may also bind to glycan structures on background glycoproteins other than the glycoprotein of interest in the lectin immunosorbant assays, (Gornik et al. 2007) resulting in a high background which jeopardizes sensitivity. This may be problematic especially when serum specimens are used because the majority of serum proteins are glycosylated.

The present invention describes the development of five lectin immunosorbant assays that are suitably analytically sensitive sensitivity and specific for the direct analysis of PSA sialylation in serum, by reducing the high background, increasing binding specificity, and using a sensitive method of detection. In the assays that have been described herein, total or free PSA antibody used to capture PSA from serum samples is oxidized in situ with 20 mM sodium periodate, which selectively destroys the carbohydrate structures on the antibody and prevents the binding of lectins to its glycans, and leaves the antibody's binding capability intact. (Gornik et al. 2007). In addition, the high background signal from binding of lectins to the glycans of background glycoproteins is reduced by adding 1% BSA into the detection buffer. In order to increase binding specificity, biotinylated lectins and the streptavidin SULFO-TAG were mixed together in the detection buffer rather than used in separate steps to prevent prolonged washing that could decrease the binding of lectins due to their low binding affinities. Finally, we used electrochemiluminescence in the MSD platform to increase the sensitivity of detection method.

The analytical advantages of these assays are multi-fold. First, using 96-well plates, these assays are high-throughput and it is possible to analyze hundreds of samples within a day. Second, rather than comparing the PSA sialylation in prostate cancer sera to that in seminal fluid, like Tabards et al did using oligosaccharide profiling by mass spectrometry, (Tabares et al. 2006) these assays have the sufficient limit of detection (0.04-1.35 ng/mL) to analyze PSA sialylation in non-cancer sera with less than 10 ng/mL of PSA and to compare them to their prostate cancer sera counterparts. Finally, they detect PSA sialylation in serum directly, as opposed to lectin affinity chromatography, which measures it indirectly. (Ohyama et al. 2004). As a result of these features, these five lectin immunosorbant assays are excellent tools for the clinical investigation of the potential role of glycoprotein analysis in improving PSA's cancer specificity.

Our results from the pooled sera study showed that α2,3-linked and α2,6-linked sialylation of PSA are more heterogeneous in cancer than in non-cancer. As noted above, it should be pointed out that the experiments can be carried out equally effectively using pooled sera or using serum samples from an individual (i.e. not pooled). This observation is consistent with findings from glycan structure analysis that PSA from prostate cancer is a mixture of biantennary, triantennary, and possibly tetraantennary oligosaccharides rather than normal PSA which has only biantennary oligosaccharides, which supports the hypothesis that oncogenic transformation of prostate epithelium may differentially affect N-linked glycan processing of PSA. (Prakash et al. 2000). In addition, the α2,3-linked sialylation patterns assessed by MAL I and MAL II were very similar, which indicates that MAL I and II may bind to the similar carbohydrate structures on PSA.

Evaluation of the clinical performance of these five lectin immunosorbant assays revealed that α2,6-linked sialylation of total PSA may be a better predictor of prostate cancer than free PSA in the 10-20% range.

Although a previous report by Ohyama et al showed that SNA bound fraction of total PSA cannot differentiate prostate cancer from BPH, our study showed it to be promising. The differences could be due to the type of specimens, the method employed as well as our focus on clinically relevant patients with % free PSA in the diagnostic gray zone. Our study used specimens with equivalent total PSA concentrations in the cancer case and non-cancer control groups, whereas Ohyama et al used specimens with total PSA concentrations in the cancer group which were much higher than in the non-cancer group (mean total PSA concentrations: 89 ng/mL vs. 8.8 ng/mL). (Ohyama et al.). This particular difference may result in the presence of different forms of glycosylated PSA in the cancer groups, because high levels of PSA are usually associated with large volume and high grade cancers, which may produce different forms of glycosylated PSA than small volume and low grade cancers that are associated with low levels of PSA. In addition, Ohyama et al used lectin affinity chromatography followed by immunodetection of PSA. Chromatographic separation of glycosylated PSA may result in the detection of different forms of glycosylated PSA than the ones detected by lectin immunosorbant assays. These differences may also explain why our MAL II assays fail to differentiate prostate cancer from non-cancer whereas their results illustrated the opposite.

The results presented herein also suggest that an assay for α2,6-linked sialylation of total PSA improves the detection of prostate cancer compared to % free PSA in its diagnostic gray zone (% free PSA=10-20%) both in an initial study in 21 subjects and in a separate study with 16 subjects. Immunosorbant assays using lectins that recognize other carbohydrate moieties (e.g., fucose) are also envisioned. These assays may also be useful in understanding perturbed glycosylation in tumor genesis and progression and could be used clinically to improve the differentiation of prostate cancer from non-cancer patients.

### Example 2

### Glycosylation Pattern Analysis of Candidate Glycoproteins from Clinical Specimens

In this study, PSA was selected as a model protein to establish a sensitive and high throughput analysis for glycosylation pattern profiling. To investigate the differential glycosylation patterns of PSA from normal and cancer patients, PSA proteins were first extracted from normal and cancer tissue samples. The PSA proteins were adjusted to same amount and profiled by a high-density lectin microarray to globally detect PSA carbohydrate patterns. The lectins which showed different signals between normal and cancer groups were selected as target marker candidates. To quantitatively analyze the glycan-lectin interactions, the ECL-based ultra-sensitive lectin-antibody immunoassays were developed to analyze targeted PSA glycan-lectin bindings at ng/mL level in clinical samples. An additional set of pooled normal and cancer tissue samples was used to validate the analytical result of lectin microarray study using the developed lectin-antibody immunoassays. Again, it should be pointed out that the experiments can be carried out equally effectively using pooled sera or using serum samples from an individual (i.e. not pooled).

### Detection of Glycosylation Patterns of Target Glycoproteins Using High-Density Lectin Microarray

To determine the sensitivity of the high-density lectin microarray, a high-density lectin microarray was used to profile different amount of PSA. Ninety-four lectins were immobilized on the glass slide using NHS eater chemistry. Each lectin was serial diluted to 4 gradients and printed in duplicate at each concentration, as shown in Table 4, below. Table 4 shows the list of detectable lectins binding to PSA from prostate tissues and serum using lectin microarray (the number represents the signal to noise ratio, while signal is the binding of the specific lectin to PSA and the noise is the same lectin without PSA).

TABLE 4

| | 1^{st} screening | | | 2^{nd} screening | | |
|---|---|---|---|---|---|---|
| | Normal | Cancer | Cancer | Normal | Cancer | Cancer |
| Lectin code | Tissue | Tissue | Serum | Tissue | Serum | Serum |
| Jacalin | — | 1.73 | 6.14 | 2.80 | 3.55 | 5.06 |
| NPA | 2.00 | 2.19 | 2.06 | 2.83 | 2.83 | 2.28 |
| LcH | 1.95 | 4.41 | 2.52 | 22.09 | 26.36 | 6.80 |
| LcH A | 5.45 | 11.02 | 16.25 | 37.90 | 35.80 | 5.12 |
| IRA | — | 1.79 | 2.53 | 5.62 | 9.84 | 2.57 |
| MPA | — | — | 8.42 | 1.63 | 3.17 | 4.28 |
| SNA-I | — | 2.21 | 20.95 | 11.19 | 16.42 | 24.20 |
| STL, PL | 30.61 | 2.42 | 27.09 | — | 4.55 | — |
| VVA | 1.59 | 2.28 | — | — | 2.88 | — |
| mannose | | | | | | |

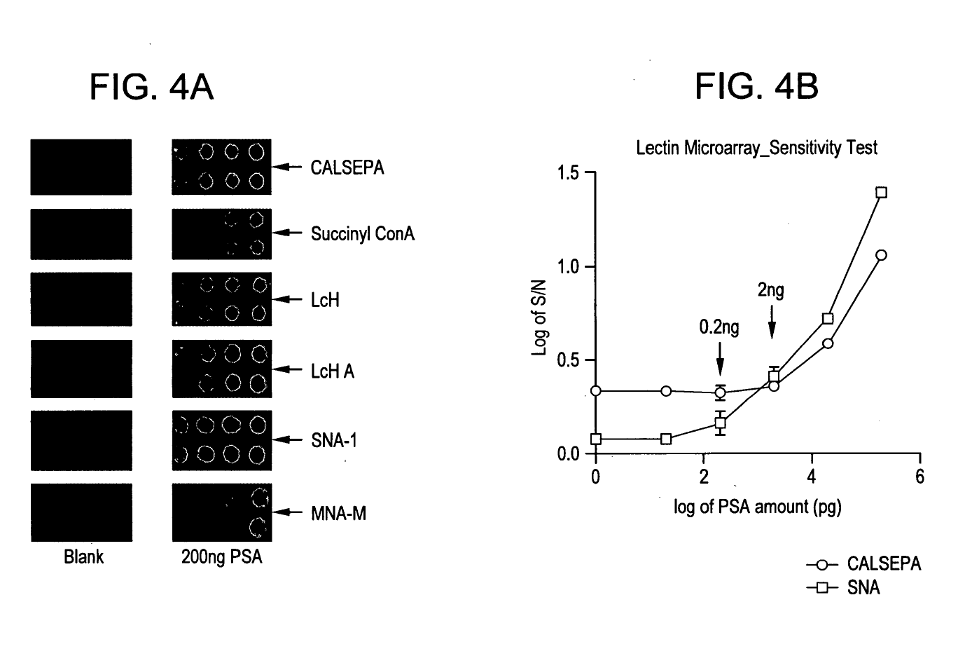

The detection sensitivity was a critical issue for the glycosylation analysis since the glycan-lectin binding is not as specific as antigen-antibody binding. To increase the detection specificity, an additional oxidation treatment of the first and second antibodies was preferably used to break cis-diol groups of sugars and avoid the interaction between the immobilized lectins and the antibodies. FIG. 4A shows the negative (TBST buffer) and positive (200 ng PSA protein) tests of the lectin microarray. The low signal of negative slide demonstrated that the lectin microarray has low background noise. The lectin signals were only observed after adding PSA protein, which indicates that the PSA glycans were specifically bonded to lectin spot. The criteria of detectable signal were set as: (1) the S/N ratio of lectin spot >1.5; (2) the ratio of sample (S/N ratio of seminal fluidic PSA) to blank (S/N ratio of negative test) of the certain lectin >1.2. For all detectable lectin spots, the signals were associated with PSA amount and were increased with higher PSA level. The Limit of Detection (LOD) of some glycan-lectin bindings were demonstrated as: 0.2 ng PSA of glycan-lectin binding for SNA-1; 2 ng PSA for CALSEPA and LcH A; 20 ng PSA for LcH; and 200 ng PSA for Succinyl ConA and MNA-M. The glycan-lectin binding curves of two lectins: SNA-1 and CALSEPA are shown as examples in FIG. 4B.

Glycosylation Profiling of PSA Proteins Extracted from Clinical Samples Using High-Density Lectin Microarray

PSA proteins were first extracted from pooled clinical samples: normal prostate tissue (N-T_{—}1, N-T_{—}2) and prostate cancer tissue (C-T_{—}1 C-T_{—}2) using PSA immunoprecipitation. To enhance the detection signal, 40 ng of PSA extracted from each sample was probed with lectin microarray. A blank sample without PSA was used as negative control. The same criteria described as above were used to distinguish the dateable signal. The lectins which have detectable signals on both arrays were listed at the Table 1. Then, the lectin signals from normal and cancer tissues were compared. Three lectins, SNA-1, Jacalin, and LcH, have been shown to be up-regulated at cancer groups in the lectin microarray in the both C-T_{—}1 and C-T 2 tissue samples, as shown in Table 5, below.

TABLE 5

| | Cancer tissue/ | Cancer tissue/ |
|---|---|---|
| | Normal tissue¹ | Normal tissue² |
| | 1st time | 2nd time |
| Lectin code | Ratio 1 | Ratio 2 |
| Jacalin | 1.25 | 1.27 |
| LcH | 2.26 | 1.20 |
| SNA-I | 1.52 | 1.47 |

¹Ratio 1 = (40 ug of PSA extracted from prostate cancer tissue sample C-T_1):(40 ug of PSA extracted from healthy prostate tissue sample N-T_1)
²Ratio 2 = (40 ug of PSA extracted from prostate cancer tissue sample C-T_2):(40 ug of PSA extracted from healthy prostate tissue sample N-T_2)

This data indicated that total PSA protein has more sialic acid, O-linked Galactose, and Man/GlcNAc core with Fuca 1-6 groups corresponding to lectin SNA-1, Jacalin, and LcH in cancer tissue. Furthermore, 40 ng of PSA extracted from pooled prostate cancer serum (C-S_{—}1, C-S_{—}2) was probed using the lectin microarray followed the processing procedures. Due to ultra-low PSA amount in the benign prostatic hyperplasia sera (BPH) and normal sera, a sufficient quantity of PSA proteins could not be collected from BPH or normal sera as control group. Therefore, a direct comparison of the carbohydrate patterns between cancer and benign/normal sera using lectin microarray was not possible. However, the detectable lectin signals in cancer sera provided useful information. The detectable lectin signals in sera have the potential to be selected as novel serum marker for cancer detection, and accordingly, the serum detectable glycan-lectin bindings have provided a candidate pool for serum marker discovery. Overall, three lectins were selected as targeted candidates for further validation study. All of them have shown different expression patterns between cancer and normal tissue, and were detectable in serum samples. These carbohydrates have the potential to become candidate glycan markers to distinguish prostate cancer from normal, and will be validated using an ultra-sensitive immunoassay in the validation study.

### Development of Ultra-Sensitive Electrochemiluminsecent-Based Immunoassays for Targeted Glycan-Lectin Analyses

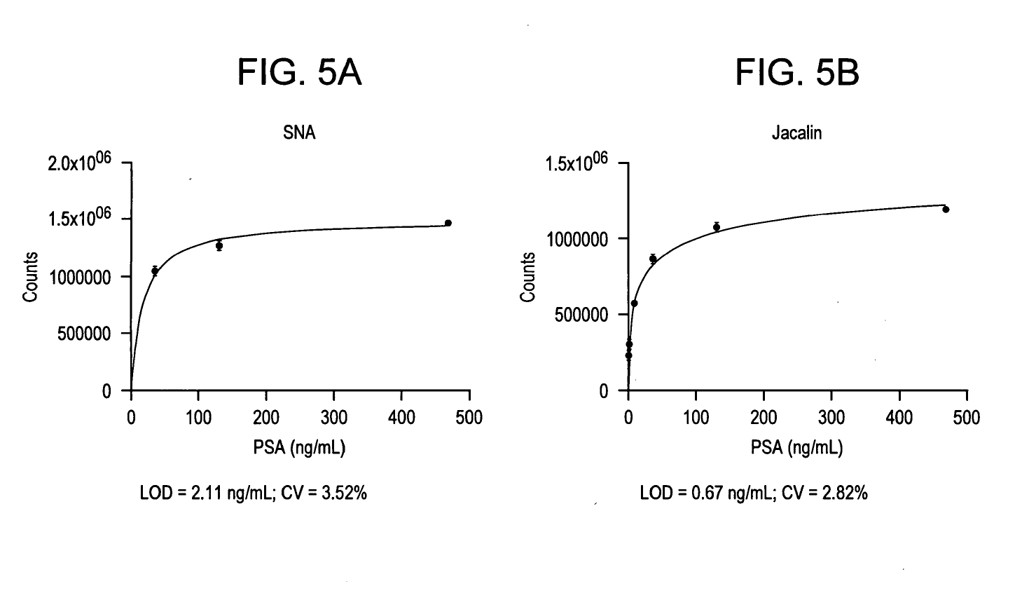

To quantitatively measure the glycan-lectin binding ratios, the electrochemiluminsecent (ECL)-based immunoassays were applied to develop ultra-sensitive analyses to detect PSA glycosylation patterns in crude clinical specimens. PSA proteins extracted from cancer sera were spiked into pooled healthy woman sera with different amounts. The final PSA concentrations were from 469.3, 129.7, 36.1, 8.8, 2.56, 0.69, to 0.19 ng/mL measured using clinical PSA assay. The PSA protein was first captured by PSA monoclonal antibody from complex clinical mixture, then coupled with lectins which has pre-labeled with biotin tag. A streptavidin conjugated with ECL-detection agent was recognize biotin tag. The chemiluminescent signal was observed when detection voltage was applied on the ECL plates after adding reading buffer. The electrochemiluminescent signals were increased with increasing amount of spike-in PSA protein in both the SNA and Jacalin assays. The LcH assay was unable to develop since it was not possible to obtain Biotinylated LcH from commercial source. The glycan-lectin binding curves were matched using special binding mode with Hill-slope to calculate the statistics parameters. The LODs and CVs of SNA and Jacalin bindings are shown in FIG. 5. FIG. 5 shows that these developed immunoassays are compatible to analyze glycosylation patterns at PSA diagnostic gray zone (4-10 ng/mL) with a good reproducibility.

### Validation of Targeted Lectin-Glycan Binding Using Developed ECL-Based Immunoassay in the Prostate Cancer Tissue Samples

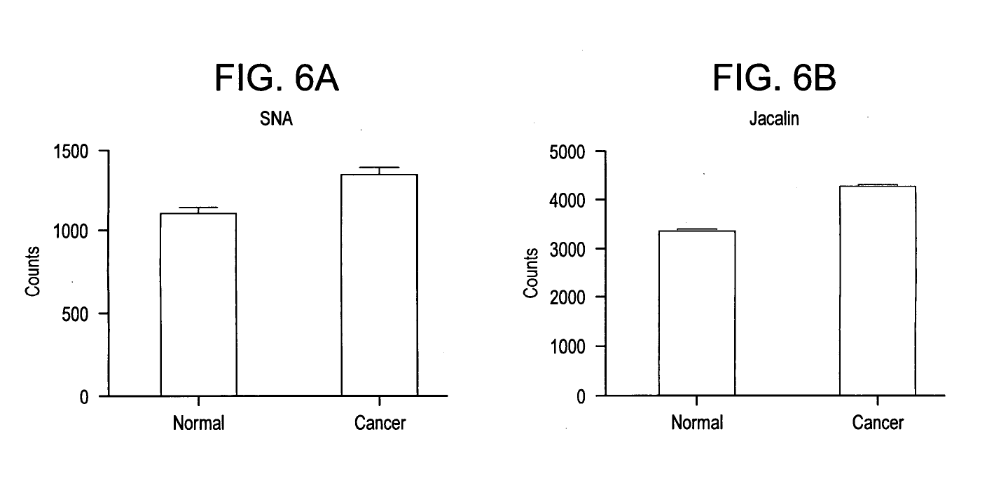

To validate the targeted glycan-lectin interactions using developed ECL-based immunoassay, an additional set of pooled normal and cancer tissue samples were prepared from different patient specimens. Both pooled samples were diluted using 1×TBST buffer to make the PSA concentrations at similar level at normal and cancer groups. Both SNA and Jacalin immunoassays have shown that the pooled prostate cancer tissue samples have higher signal than normal tissues. These results were agreeable with the analytical results of lectin microarray study, as shown in FIG. 6.

Protein glycosylation is one of the most common protein modifications of proteins expressed in the extracellular environment, including membrane proteins, cell surface proteins, and secreted proteins. These protein are among the most accessible proteins for therapeutic or diagnostic purposes. Moreover, the FDA (Food and Drug Administration)-approved tumor protein markers are all glycoproteins. To increase the detection power of glycoprotein markers, such as PSA for prostate cancer diagnosis, the present study of carbohydrate expression of marker proteins was provided as a method to improve cancer detection. Most of the FDA-approved markers are directed to low abundance proteins in clinical sample. Further, it can be difficult to collect enough low abundance protein from clinical specimens for carbohydrate analysis using conventional chromatography or electrophoresis methods. For example, most of the published papers have to compare the glycan patterns of PSA protein from non-clinical source, such as using PSA from seminal fluid vs. PSA collected from prostate cancer cell line. It is not surprising that the results that have been reported do not represent the clinical status of PSA glycan pattern since the PSA carbohydrates were expressed in different cell culture environments. Accordingly, glycosylation expression studies are preferably directly linked to clinical specimens for clinical application.

Considering the low abundance of marker protein in clinical samples, the detection sensitivity is the first consideration for carbohydrate profiling. To reduce the LOD down to ng or ng/mL level, the experimental procedures described herein were optimized for both analyses. For the lectin microarray detection, the PSA protein was first extracted from clinical sample to avoid the interactions between lectins and glycans from other proteins. Then, PSA antibody was added to complete a sandwich ELISA to increase detection specificity. The PSA antibody and fluorescent label were oxidized to break cis-diol group of sugar, thus reduced the interaction between lectins and the glycans from these proteins. By combined all the treatments, the LODs of some lectins in the lectin microarray were reduced to 0.2 ng and 2 ng of PSA. For the ECL-based lectin-antibody immunoassay, the PSA antibody coated on the MSD plate was treated with peroxide to break the sugar group.

Electrochemiluminescent (ECL) detection is an ultra-sensitive analytical method comparing to conventional ELISA. In an ECL detection, the instrument measured the emit light from the ECL-labeled detection agents when reading voltage was applied on the plate. No excitation light source caused additional background noise for detection. Additionally, only the antibody-captured PSA-lectin complex which was near the electrode surface (bottom of the plate) was able to be detected. The non-specific binding proteins which attached on the well wall cannotobtain electric energy and generate noise at this system. Both of the lectin microarray and the ECL-detection were able to analyze PSA glycosylation patterns down to ng or ng/mL of PSA with suitably good reproducibility in this study.

Detection throughput is another consideration for clinical detection. Lectin microarray was able to profile hundreds of lectins in one single test. However, it required additional treatment of clinical samples. The targeted protein had to been isolated from clinical sample to avoid the interference between lectins and glycans from other tissue or serum proteins. It was a great tool for pre-screening, but may not be suitable to profile glycosylation expression for a large set of clinical samples. The ECL-based immunoassay had a similar format to the conventional ELISA. The PSA protein was able to be captured from complex clinical samples by PSA antibody coated in the MSD plate. No addition sample treatment was needed. Clinical specimens can be directly added to the plate at this detection platform. Accordingly, it allows for the high throughput detection to analyze the large amount of clinical samples.

Preferably, in order to compare the glycosylation patterns between normal and cancer clinical samples, quantitative analyses was preferably established. The lectin microarray can not suitably provide an absolute quantitative analysis since the lectin spot only holds certain amount of lectin molecules. Once the PSA protein amount exceeded the immobilized lectin amount, especially at the low lectin concentration spot, the spot was saturated and cannot represent the real PSA level. Accordingly, the ultra-sensitive ECL-based immunoassays for targeted glycan-lectin bindings were suitably developed and the lectin microarray results were validated using additional set of clinical samples.

The data and results described herein describe a two-phase analytical platform which combine a high-density lectin microarray and ECL-based lectin-antibody immunoassay to investigate glycosylation patterns in clinical specimens. A large amount of lectins were suitably profiled with targeted protein from cancer patients to pre-screen targeted glycan-lectin bindings. The ECL-based immunoassay was preferably developed for selected lectin targets and the lectin microarray results were validated using additional set of pooled samples. This method has been used to profile the glycosylation change of PSA protein from prostate normal and cancer tissue samples. Lectin SNA and Jacalin have shown up-regulated signals in cancer samples, and have been validated using ECL-immunoassays. Even without a detailed glycan structure, this two-phase analytical platform can provide cancer-related information in a precise, ultra-sensitive, reproducible, and high-throughput way for glycosylation pattern profiling in clinical specimens.

### Methods

The present invention was performed with, but not limited to, the following methods.

### Human Serum Samples

Individual serum samples were obtained from 26 patients with biopsy-confirmed prostate cancer and 26 patients with biopsy-confirmed non-cancer prior to biopsy. Total PSA concentrations of the non-cancer and cancer groups were matched so that the great majority (87%) had total PSA concentrations between 4 and 10 ng/mL. In addition, 3 prostate cancer serum pools and 3 non-cancer serum pools were prepared from patients with and without prostate cancer, respectively. Both the total and free PSA concentrations of these 6 pools were matched so that their total PSA concentrations were in the range of 5-6 ng/mL and free PSA concentrations were in the range of 0.8-1.6 ng/mL. The 16 subjects (8 cancer and 8 non-cancer) used in a separate study to validate the clinical performance of total SNA assay had total PSA concentrations in the range of 3.1-10.4 ng/mL and % free PSA in the range of 9.2-20.3%.

### Reagents

MESO SCALE DISCOVERY (MSD) 96-well standard plates, MSD SULFO-TAG, and MSD plate read buffer T (4×) were from Meso Scale Discovery (Gaithersburg, Md.). Total and free PSA monoclonal antibodies (Clone BP001 and AP003S) were from Scripps Laboratory. Human PSA (100% free PSA from human seminal fluid was from Lee Biosolutions, Inc (St. Louis, Mo.). Biotinylated sambucus nigra lectin (SNA), biotinylated maackia amurensis lectin I (MAL I), biotinylated maackia amurensis lectin II (MAL II) were from Vector Laboratories (Burlingame, Calif.). Bovine serum albumin (BSA) and Tween 20 were from Sigma-Aldrich (St. Louis, Mo.). 10× Tris buffered saline (TBS) was from Bio-Rad (Hercules, Calif.).

### Lectin Immunosorbant Assays

MSD plates were coated with 30 μL of the PSA monoclonal antibody at a concentration of 7.5 ug/mL and incubated at 4° C. overnight. Unbound antibody solution was discarded and 150 μL of TBS buffer with 5% BSA was used for blocking at room temperature (RT) for 1 hour with shaking. Next, plates were washed three times using TBS+0.1% (v/v) Tween 20. In order to prevent binding of lectins to the carbohydrate determinants on the PSA antibody, antibody coated on the plates was treated with 150 μL of sodium periodate buffer prepared in 150 mM NaCl and 100 mM sodium acetate (pH 5.5) at 4° C. for 1 hour. 14, 15 Following treatment, the plates were washed as before and 50 μl of serum sample was added to each well and incubated at RT for 2 hours with shaking. Plates were washed 10 times with TBS+0.1% Tween 20 buffer and 25 μL of the detection buffer containing 80 μM biotinylated lectin (e.g. SNA, MAL I or MAL II) and 5 μM MSD streptavidin SULFO-TAG was added to each well for incubation at RT for 1 hour. Finally, 150 μL of 1×MSD plate read buffer was added to each well for electrochemiluminescence (ECL) detection using the MSD SECTOR Imager 2400.

### Evaluation of Analytical Performance

Pooled female sera spiked with various concentrations of human seminal fluid PSA (final concentrations: 0.01, 0.76, 2.34, 7.05, 23.03, and 46.86 ng/mL) were used to develop the assays. The limit of detection (LOD) was calculated based on the signal of the background (0 ng/mL concentration) plus 3 times the standard deviation (SD) of the background. Total and free PSA concentrations in these pools were the same. Within-run reproducibility (n=27) was assessed using pooled male sera at two levels of endogenous total PSA (4.12 ng/mL and 11.22 ng/mL) and free PSA (0.91 ng/mL and 0.99 ng/mL). The total and free PSA concentrations in these samples were determined using the Beckman ACCESS Hybritech PSA and Free PSA assays, respectively.

### Data Analysis

PSA glycosylation results from these five lectin immunosorbant assays were expressed in electrochemiluminescence intensity. The Mann-Whitney U-test was used to compare differences between the study groups. The statistical software MedCalc was used to construct ROC curves and to calculate their areas and confidence intervals (CIs).

### Materials

Nexterion H Slide was purchased from SCHOTT North America Inc. (Lousville, Ky.). Ninety-four lectins were provided by Dr Heng Zhu and collected from 4 commercial sources (as shown in Table 4). Human PSA from seminal fluid was from Lee BioSolutions, Inc. (St. Louis, Mo.). Mouse anti-human PSA antibody was from Scripps Laboratories (San Diego, Calif.). Rabbit anti-mouse IgG-Alexa Fluor 647 conjugate was from Invitrogen (Eugene, Oreg.). Non-protein blocker was from Thermo Fisher Scientific Inc. (Rockford, Ill.). Incubation chamber and holder for lectin microarray were from Whatman Schleicher & Schuell (Keene, N.H.). Anti-human PSA (total) antibody-coated magnetic beads was from Beckman Coulter Inc. (Fullerton, Calif.). Electrochemiluminescent assay including MESO SCALE DISCOVERY (MSD) 384-well standard plates, blocker kit, MSD SMLFO-TAG, MSD plate read buffer T (4×) were purchased from Meso Scale Discovery (Gaithersburg, Md.). Rabbit anti-human PSA antibody was from Affinity Bioreagents (Golden, Colo.). Sodium periodate was from Bio-Rad Laboratories (Hercules, Calif.). Biotinylated Jacalin and Biotinylated Sambucus Nigra Lectin (SNA) were from Vector Laboratories (Burlingame, Calif.). All other chemicals and reagents were purchased from Sigma-Aldrich (St. Louis, Mo.).

### Pooled Prostate Cancer Tissue

N-T_{—}1, N-T_{—}2, N-T_{—}3, pooled healthy prostate tissue C-T_{—}1, C-T_{—}2, C-T_{—}3, and pooled prostate cancer sera C—S_{—}1, C-S_{—}2, were prepared by the Clinical Chemistry Laboratory at Johns Hopkins University. The PSA concentrations were measured using the Beckman ACCESS Hybritech PSA assays.

### Lectin Microarray Fabrication and Quality Control

Lectin proteins were resuspended in a phosphate buffered saline (PBS) buffer with 0.02% Tween20 and 25% glycerol to a final concentration of 1 μg/μL. Bovine serum albumin (BSA, 0.05 μg/μL) was also added to the buffer to improve spot morphology. The lectins were printed on Nexterion H Slides using the ChipWriter Pro (Bio-Rad, Hercules, Calif.) microarrayer. The lectins with four concentrations were printed in duplicate at each block and 6 sets of lectin blocks were printed per slide. After printing, slides were covered with aluminum foil and stored at 4° C. for future use. To monitor the quality of lectin spotting, the microarrays were stained with 549 NHS Ester (DyLight) in 100-fold dilution at room temperature for 1 hour. The stained slides were washed twice with TBST (1×TBS+0.1% Tween-20) followed by one wash with water. The dried slides were scanned with a GenePix 4100B (Axon, Sunnyvale, Calif.) scanner at 10 μm resolution. The scanning conditions were 600 mV laser power and 33% PMT value at the Cy3 channel.

### The Sensitivity Test of the High-Density Lectin Microarray Using Seminal Fluidic PSA

The lectin microarray was integrated with incubation chamber and array holder to probe PSA samples by using the following procedures. First, lectin microarray was immersed into 50 mM ethanolamine in borate buffer (pH 8.0) for 1 hour for surface blocking. Blocked slide was washed once using TBST buffer, followed by water. Slide was dry out by spinning at 500 g for 5 min. Second, 0 ng, 0.02 ng, 0.2 ng, 2 ng, 20 ng, and 200 ng of PSA protein isolated from seminal fluid were diluted into 200 μL using 1×TBST buffer. The samples were added to each set of lectin block, and incubated at room temperature (RT) for 2 hours with gentle shaking. The microarray was then rinsed with 200 μL of 1×TBST buffer to remove non-binding proteins for three times. Third, the first antibody (mouse anti-human PSA antibody) and the second antibody (rabbit anti-mouse IgG-Alexa Fluor 647 conjugate) were mixed with 20 mM sodium periodate to oxidize cis-diol bond of sugar group at 4° C. for 1 hour in the dark. The 200 μL of 2 μg/mL oxidized mouse anti-human PSA antibody was hybridized with the microarray for 1 hour with gentle shaking. Additional washing was used to remove the free antibodies. Fourth, 200 μL of 2 μg/mL oxidized rabbit anti-mouse IgG-Alexa Fluor 647 conjugate was added as the second antibody and hybridized with the microarray for 1 hour with gentle shaking. After TBST buffer washing, the microarray was released from incubation chamber and washed by H2O twice. The array was dried out by spinning at 500 g for 5 minutes, then immediately scanned by a GenePix 4000B scanner at wavelength of 647 nm and PMT setting 800. The slide images were analyzed using GenePix 3.0 software to convert to numerical format (GPR) using a homemade “.GAL” file. Medium of spot foreground intensity, medium of spot background intensity, and the lectin protein identification were used in this analysis. The SN ratio (the medium of spot foreground intensity to the medium of spot background intensity) of each lectin spot was used to analyze the Limit of Detection (LOD) of each lectin.

Glycan Profiling of PSA Extracted from Clinical Samples Using High-Density Lectin Microarray

50 μL of each pooled clinical samples (N-T_{—}1, N-T_{—}2, C-T_{—}1, C-T_{—}2, C—S_{—}1, C-S_{—}2) was incubated with 100 μL of anti-human PSA (total) antibody-coated magnetic at 4° C. for 12 hours. The beads were washed six times by 1×TBST buffer. 100 μL of 100 mM glycine (pH 2.3) was used to elute PSA protein from magnetic beads for three times. The eluted solutions were collected and adjusted pH to 7.5 using 30 μL of 10×TBST buffer and 5 μL˜10 μL of 30% NaOH. The final PSA concentration was measured using the Beckman ACCESS Hybritech PSA assays. 40 ng of PSA protein of each pooled clinical samples was diluted to 200 μL of 1×TBST buffer. The PSA samples were incubated with lectin microarray using the protocol described above. 2004 of 1×TBST buffer without PSA protein was used as negative control in this test. The SN ratios of clinical PSA protein divided by the SN ratios of blank array of corresponded lectin spots were used for data analysis.

### Ultra-Sensitive Electrochemiluminsecent-Based Immunoassay for Targeted Glycan-Lectin Interactions of PSA Protein

To quantitatively detect the glycan-lectin interactions, the ultra-sensitive immunoassays for targeted lectins were established using ECL-based analyses. 384-MSD plate was first coated with 10 μL of 10 μg/mL mouse anti-human PSA antibody overnight at 4° C. Then the MSD plate was blocked using 50 μL of non-protein blocker at RT for 1 hour with gentle shaking. The plate was washing three times using 1×TBST buffer. To reduce background from lectin-PSA antibody binding, The coated PSA monoclonal antibody was oxidized using 50 μL of 20 mM sodium periodate in 4° C. in the dark for 1 hour to break cis-diol group of sugar. The extra sodium periodate was washing away using 1×TBST buffer for three times. PSA protein extracted from pooled prostate cancer sera was diluted using pooled healthy woman sera to generate concentration gradient from 1000 ng/mL to 0.244 ng/mL using 4× dilution. Final PSA concentrations were measured using the Beckman ACCESS Hybritech PSA assays. 10 μL of samples were incubated in triplicate into MSD wells at RT for 2 hours with gentle shaking. Non-bonded proteins were washed away using TBST buffer for three times. Then 104 of 20 μg/mL of biotinylated lectin and 5 μg/mL of streptavidin SMLFO-TAG were added to each well at RT for 1 hour incubation with gentle shaking. The extra lectin and SMLFO-TAG were washing away using 1×TBST buffer for three times. Finally, 50 μL of 1×MSD read buffer was added to each well and read immediately using the MSD SECTOR Imager 2400. The data was analyzed using Prism software to analyze the detection sensitivity for each glycan-lectin binding.

### Validation Study Using Developed Electrochemiluminsecent-Based Immunoassays for Targeted Glycan-Lectin Interactions of PSA Protein in Prostate Cancer Tissues

The pooled prostate cancer tissue (C-T_{—}2) and normal tissue (N-T_{—}2) samples were first diluted using 1×TBST buffer to adjust the total PSA protein at same level. The total PSA concentrations were measured using the Beckman ACCESS Hybritech PSA assays and final concentrations were: 162.20 ng/mL in N-T-2 sample and 163.36 ng/mL in C-T_{—}2 sample. The 1×TBST buffer was used as negative control. The ECL-based antibody-lectin immunoassay procedure was described at above. In briefly, 10 μL of each sample was added in triplicate into 384-MSD plate after plate blocking and oxidization. Then 10 μL of 20 μg/mL of biotinylated Jacalin or SNA was mixed with 5 μg/mL of streptavidin SMLFO-TAG to probe with PSA protein individually. 50 μL of 1×MSD plate read buffer was added to each well for electrochemiluminescence detection using the MSD SECTOR Imager 2400. The data was analyzed using Prism software to analyze the different glycan-lectin binding ratios between normal and cancer groups.

### REFERENCES

Catalona, W. J.; Smith, D. S.; Ratliff, T. L.; Dodds, K. M.; Coplen, D. E.; Yuan, J. J.; Petros, J. A.; Andriole, G. L., Measurement of prostate-specific antigen in serum as a screening test for prostate cancer. N Engl J Med 1991, 324, (17), 1156-61.
Thompson, I. M.; Pauler, D. K.; Goodman, P. J.; Tangen, C. M.; Lucia, M. S.; Paynes, H. L.; Minasian, L. M.; Ford, L. G.; Lippman, S. M.; Crawford, E. D.; Crowley, J. J.; Coltman, C. A., Jr., Prevalence of prostate cancer among men with a prostate-specific antigen level < or =4.0 ng per milliliter. N Engl J Med 2004, 350, (22), 2239-46.
Chan, D. W.; Bruzek, D. J.; Oesterling, J. E.; Rock, R. C.; Walsh, P. C., Prostate-specific antigen as a marker for prostatic cancer: a monoclonal and a polyclonal immunoassay compared. Clin Chem 1987, 33, (10), 1916-20.
Catalona, W. J.; Richie, J. P.; Ahmann, F. R.; Hudson, M. A.; Scardino, P. T.; Flanigan, R. C.; deKernion, J. B.; Ratliff, T. L.; Kavoussi, L. R.; Dalkin, B. L.; et al., Comparison of digital rectal examination and serum prostate specific antigen in the early detection of prostate cancer: results of a multicenter clinical trial of 6,630 men. J Urol 1994, 151, (5), 1283-90.
Lilja, H.; Christensson, A.; Dahlen, U.; Matikainen, M. T.; Nilsson, O.; Pettersson, K.; Lovgren, T., Prostate-specific antigen in serum occurs predominantly in complex with alpha 1-antichymotrypsin. Clin Chem 1991, 37, (9), 1618-25.
Brawer, M. K., Prostate-specific antigen: current status. CA Cancer J Clin 1999, 49, (5), 264-81.
Mikolajczyk, S. D.; Marks, L. S.; Partin, A. W.; Rittenhouse, H. G., Free prostate-specific antigen in serum is becoming more complex. Urology 2002, 59, (6), 797-802.
Belanger, A.; van Halbeek, H.; Graves, H. C.; Grandbois, K.; Stamey, T. A.; Huang, L.; Poppe, I.; Labrie, F., Molecular mass and carbohydrate structure of prostate specific antigen: studies for establishment of an international PSA standard. Prostate 1995, 27, (4), 187-97.
Hakomori, S., Aberrant glycosylation in tumors and tumor-associated carbohydrate antigens. Adv Cancer Res 1989, 52, 257-331.
Narayanan, S., Sialic acid as a tumor marker. Ann Clin Lab Sci 1994, 24, (4), 376-84.
Durand, G.; Seta, N., Protein glycosylation and diseases: blood and urinary oligosaccharides as markers for diagnosis and therapeutic monitoring. Clin Chem 2000, 46, (6 Pt 1), 795-805.
Tabares, G.; Radcliffe, C. M.; Barrabes, S.; Ramirez, M.; Aleixandre, R. N.; Hoesel, W.; Dwek, R. A.; Rudd, P. M.; Peracaula, R.; de Llorens, R., Different glycan structures in prostate-specific antigen from prostate cancer sera in relation to seminal plasma PSA. Glycobiology 2006, 16, (2), 132-45.
Ohyama, C.; Hosono, M.; Nitta, K.; Oh-eda, M.; Yoshikawa, K.; Habuchi, T.; Arai, Y.; Fukuda, M., Carbohydrate structure and differential binding of prostate specific antigen to Maackia amurensis lectin between prostate cancer and benign prostate hypertrophy. Glycobiology 2004, 14, (8), 671-9.
Zhang, H.; Li, X. J.; Martin, D. B.; Aebersold, R., Identification and quantification of N-linked glycoproteins using hydrazide chemistry, stable isotope labeling and mass spectrometry. Nat Biotechnol 2003, 21, (6), 660-6.
Gornik, O.; Lauc, G., Enzyme linked lectin assay (ELLA) for direct analysis of transferrin sialylation in serum samples. Clin Biochem 2007, 40, (9-10), 718-23.
Knibbs, R. N.; Goldstein, I. J.; Ratcliffe, R. M.; Shibuya, N., Characterization of the carbohydrate binding specificity of the leukoagglutinating lectin from Maackia amurensis. Comparison with other sialic acid-specific lectins. J Biol Chem 1991, 266, (1), 83-8.
Kawaguchi, T.; Matsumoto, I.; Osawa, T., Studies on hemagglutinins from Maackia amurensis seeds. J Biol Chem 1974, 249, (9), 2786-92.
Peracaula, R.; Tabares, G.; Royle, L.; Harvey, D. J.; Dwek, R. A.; Rudd, P. M.; de Llorens, R., Altered glycosylation pattern allows the distinction between prostate-specific antigen (PSA) from normal and tumor origins. Glycobiology 2003, 13, (6), 457-70.
Tajiri, M.; Ohyama, C.; Wada, Y., Oligosaccharide profiles of the prostate specific antigen in free and complexed forms from the prostate cancer patient serum and in seminal plasma: a glycopeptide approach. Glycobiology 2008, 18, (1), 2-8.
Sokoll, L. J.; Wang, Y.; Feng, Z.; Kagan, J.; Partin, A. W.; Sanda, M. G.; Thompson, I. M.; Chan, D. W., [−2]proenzyme prostate specific antigen for prostate cancer detection: a national cancer institute early detection research network validation study. J Urol 2008, 180, (2), 539-43; discussion 543.
Mikolajczyk, S. D.; Catalona, W. J.; Evans, C. L.; Linton, H. J.; Millar, L. S.; Marker, K. M.; Katir, D.; Amirkhan, A.; Rittenhouse, H. G., Proenzyme forms of prostate-specific antigen in serum improve the detection of prostate cancer. Clin Chem 2004, 50, (6), 1017-25.
Barak, M.; Mecz, Y.; Lurie, A.; Gruener, N., Binding of serum prostate antigen to concanavalin A in patients with cancer or hyperplasia of the prostate. Oncology 1989, 46, (6), 375-7.
Chan, D. W.; Gao, Y. M., Variants of prostate-specific antigen separated by concanavalin A. Clin Chem 1991, 37, (6), 1133-4.
Lotan, R.; Nicolson, G. L., Purification of cell membrane glycoproteins by lectin affinity chromatography. Biochim Biophys Acta 1979, 559, (4), 329-76.
McCoy, J. P., Jr.; Varani, J.; Goldstein, I. J., Enzyme-linked lectin assay (ELLA): use of alkaline phosphatase-conjugated Griffonia simplicifolia B4 isolectin for the detection of alpha-D-galactopyranosyl end groups. Anal Biochem 1983, 130, (2), 437-44.
Mehta, A. S.; Long, R. E.; Comunale, M. A.; Wang, M.; Rodemich, L.; Krakover, J.; Philip, R.;
Marrero, J. A.; Dwek, R. A.; Block, T. M., Increased levels of galactose-deficient anti-Gal immunoglobulin G in the sera of hepatitis C virus-infected individuals with fibrosis and cirrhosis. J Virol 2008, 82, (3), 1259-70.
Davies, C., Principles. In The Immunoassay Handbook, Wild, D., Ed. Stockton Press: New York City, N.Y., 1994.
Prakash, S.; Robbins, P. W., Glycotyping of prostate specific antigen. Glycobiology 2000, 10, (2), 173-6.
Arnold, J. N., et al., The impact of glycosylation on the biological function and structure of human immunoglobulins. Annu Rev Immunol, 2007. 25: p. 21-50.
Roth, J., Protein N-glycosylation along the secretory pathway: relationship to organelle topography and function, protein quality control, and cell interactions. Chem Rev, 2002. 102(2): p. 285-303.
Lowe, J. B., Glycosylation in the control of selectin counter-receptor structure and function. Immunol Rev, 2002. 186: p. 19-36.
Hounsell, E. F., M. J. Davies, and D. V. Renouf, O-linked protein glycosylation structure and function. Glycoconj J, 1996. 13(1): p. 19-26.
Gorelik, E., U. Galili, and A. Raz, On the role of cell surface carbohydrates and their binding proteins (lectins) in tumor metastasis. Cancer Metastasis Rev, 2001. 20(3-4): p. 245-77.
Kannagi, R., et al., Carbohydrate-mediated cell adhesion in cancer metastasis and angiogenesis. Cancer Sci, 2004. 95(5): p. 377-84.
Orntoft, T. F. and E. M. Vestergaard, Clinical aspects of altered glycosylation of glycoproteins in cancer. Electrophoresis, 1999. 20(2): p. 362-71.
Dennis, J. W., M. Granovsky, and C. E. Warren, Glycoprotein glycosylation and cancer progression. Biochim Biophys Acta, 1999. 1473(1): p. 21-34.
Stephan, C., et al., PSA and other tissue kallikreins for prostate cancer detection. Eur J Cancer, 2007. 43(13): p. 1918-26.
Khan, M. A., et al., Evaluation of proprostate specific antigen for early detection of prostate cancer in men with a total prostate specific antigen range of 4.0 to 10.0 ng/ml. J Urol, 2003. 170(3): p. 723-6.
Sumi, S., et al., Serial lectin affinity chromatography demonstrates altered asparagine-linked sugar-chain structures of prostate-specific antigen in human prostate carcinoma. J Chromatogr B Biomed Sci Appl, 1999. 727(1-2): p. 9-14.
Donohue, M. J., et al., Capillary electrophoresis for the investigation of prostate-specific antigen heterogeneity. Anal Biochem, 2005. 339(2): p. 318-27.
Tao, S. C., et al., Lectin microarrays identify cell-specific and functionally significant cell surface glycan markers. Glycobiology, 2008. 18(10): p. 761-9.
Kuno, A., et al., Focused differential glycan analysis with the platform antibody-assisted lectin profiling for glycan-related biomarker verification. Mol Cell Proteomics, 2009. 8(1): p. 99-108.
Meany, D. L., et al., Glycoproteomics for prostate cancer detection: changes in serum PSA glycosylation patterns. J Proteome Res, 2009. 8(2): p. 613-9.

1. A method of determining if a subject has prostate cancer comprising:
determining if the subject has an altered PSA glycosylation pattern as compared to the glycosylation pattern of PSA from a healthy subject;
wherein an altered glycosylation pattern is indicative that the subject has prostate cancer.

2. The method of claim 1, wherein the glycosylation pattern is α2,3-linked sialylation or α2,6-linked sialylation of PSA.

3. The method of claim 1, wherein the PSA glycosylation pattern is determined by one or more lectin immunosorbant assays.

4. The method of claim 3, wherein the one or more lectin immunosorbant assays sandwich serum PSA between a PSA antibody and one or more lectins.

5. The method of claim 3, wherein the one or more immunosorbant assays are selected from the group consisting of total PSA with SNA, total PSA with MAL I, total PSA with MAL II, free PSA with MAL I and free PSA with MAL II.

6. The method of claim 5, wherein the method comprises at least 2 lectin immunosorbant assays.

7. The method of claim 5, wherein the method comprises at least 3 lectin immunosorbant assays.

8. The method of claim 5, wherein the method comprises at least 4 lectin immunosorbant assays.

9. The method of claim 5, wherein the method comprises 5 lectin immunosorbant assays.

10. The method of claim 1, further comprising isolating PSA from a biological sample using a PSA specific antibody.

11-21. (canceled)

22. A method of determining if a subject has prostate cancer, comprising:
determining if a subject has an altered PSA α2,6-sialylation pattern as compared to the α2,6-sialylation pattern of PSA from a healthy subject;
wherein an altered PSA α2,6-sialylation pattern is indicative of prostate cancer.

23. The method of claim 22, the PSA α2,6-sialylation pattern is determined by lectin immunosorbant assay.

24. The method of claim 23, wherein the lectin immunosorbant assay is an assay of total PSA with SNA.

25. The method of claim 22, further comprising isolating total PSA from a biological sample using a total PSA specific antibody.

26. The method of claim 22, wherein the subject is preselected based on the levels of free PSA.

27-33. (canceled)

34. A method of determining if a subject has prostate cancer, comprising:
determining if a subject has an altered PSA α2,3-sialylation pattern as compared to the α2,3-sialylation pattern of PSA from a healthy subject;
wherein an altered PSA α2,3-sialylation pattern is indicative of prostate cancer.

35-45. (canceled)

46. A method for determining if a subject has cancer or benign prostate hyperplasia (BPH) comprising:
determining if a subject has an altered PSA α2,6-sialylation pattern as compared to the α2,6-sialylation pattern of PSA from a healthy subject;
wherein an altered PSA α2,6-sialylation pattern is indicative of prostate cancer and a non-altered α2,6-sialylation pattern of PSA is indicative of benign prostate hyperplasia.

47-53. (canceled)

54. A method of determining if a subject has prostate cancer comprising:
determining if a sample of PSA from a subject has increased levels of glycosylation with sialic acid, O-linked galactose or Man/GlcNAc with Fuca1-6 groups as compared to PSA from a healthy subject;
wherein PSA with increased levels of glycosylation with sialic acid, O-linked galactose or Man/GlcNAc with Fuca1-6 groups as compared to PSA from a healthy subject is indicative of prostate cancer.

55. The method of claim 54, wherein the PSA glycosylation pattern is determined by one or more lectin immunosorbant assays.

56-65. (canceled)

66. A kit for determining if a subject has prostate cancer comprising one or more lectins and a PSA specific antibody and instructions for use, or
A kit for determining if a subject has prostate cancer comprising lectins SNA-1, Jacalin, and LcH, a PSA specific antibody and instructions for use

67-79. (canceled)
